(19) United States
(12) Patent Application Publication
(10) Pub. No.: US 2003/0118569 A1
(43) Pub. Date: Jun. 26, 2003

(54) Method of enhancing immunogenicity by covalent linkage of antigens to proteins on the surface of dendritic cells

(76) Inventors: Richard B. Bankert, Eden, NY (US); Nejat Egilmez, East Amherst, NY (US)

Correspondence Address: HODGSON RUSS LLP, ONE M & T PLAZA, SUITE 2000, BUFFALO, NY, 14203-2391, US

(21) Appl. No.: 10/323,338

(22) Filed: Dec. 18, 2002

Related U.S. Application Data
(60) Provisional application No. 60/342,356, filed on Dec. 18, 2001.

(51) Int. Cl. A61K 48/00; C12N 15/85; C12N 5/08

(52) U.S. Cl. 424/93.21; 435/372; 435/455

(57) ABSTRACT

A method is provided to increase the immunogenicity of an antigen. This method involves the covalent coupling of the antigen to proteins or glycoproteins present on the surface of dendritic cells by a mild biochemical modification which minimizes the denaturation of the antigen and preserving cell viability. Dendritic cells with covalently linked antigen on their surface can be used for generating a specific response to the antigen. The present method can be used for both therapeutic and preventive purposes.

This application claims priority to U.S. provisional application No. 60/342,356, filed on Dec. 18, 2002, the disclosure of which is incorporated herein by reference.

This invention was made with government support under grant no. CA 79879 from the National Cancer Institute. The government has certain rights in the invention.

### FIELD OF THE INVENTION

The present invention relates to the general field of immunotherapy and more particularly provides a method for increasing immunogenicity of an antigen.

### BACKGROUND OF AN INVENTION

Dendritic cells (DCs) are unique among antigen presenting cells (APCs) in their ability to stimulate naïve T cells and initiate primary immune responses (Steinman 2000). The combination of several properties found in DCs enables these cells to serve as the initiators of immunity. DCs are located throughout the body and are most highly concentrated at the organism's interface with the environment (i.e. epidermis and dermis and the mucosal surfaces of the lung and gastrointestinal tract). It is here that DCs are considered to survey their surroundings and through the production of soluble factors, alert innate effectors to invasion by pathogens. Upon interaction with microbial products or inflammatory cytokines, DCs produce chemokines and cytokines that recruit and activate additional APCs and immune effector cells. In addition to mobilizing the innate response, DCs collect information from their microenvironment and serve as liaisons between the peripheral tissues and the naïve T cells, which are limited to passage through blood and lymphoid organs. The DCs, carrying antigen (Ag) encounter potentially receptive T cells in peripheral lymphoid tissues. DCs have the ability to direct an effective adaptive immune response. DCs deliver information in the form of foreign peptide bound to MHC molecules in concert with costimulatory molecules and soluble cytokine release to prime and direct the generation of a T cell response. Through their ability to direct the initiation of both the innate and adaptive immune responses DCs play an invaluable role in immune defense.

A system has developed in DCs which directs a host organism's response toward cellular (T_{H}1) or humoral (T_{H}2) immunity. In the act of initiating an immune response DCs convey information about their sentinel experience in the form of three instructive signals. The first signal advises of foreignness of a peptide in the context of MHC. The second signal conveys the presence or absence of danger through the expression of costimulatory molecules. The third signal supplies information (gained through evolution) that directs the type of immune response that should be mounted.

An emerging example of the delivery of a third signal by DCs can be found by examining the type of immune response induced in the presence of bacterial DNA. Bacterial DNA contains motifs that include unmethylated Cytidine-phosphate-Guanosine (CpG) repeats. The CpG repeats and their specific flanking sequences impart a potentiating activity to the bacterial DNA (Kreig et al. 2000). The immunological effects of bacterial CpG containing oligonucleotides are profound. Unmethylated bacterial CpG repeats bind to a receptor, toll like receptor-9 (TLR-9) on DCs and induces maturation of immature cells. Not only do these products cause maturation of DCs in terms of increasing surface expression of costimulatory molecules and MHC products, but these products also allow for the delivery of a T_{H}1, or cellular immune response biasing third signal. CpG oligonucleotides induce the third signal by causing DC production of IL-12 (Sparwasser 1998, Brunner et al. 2000).

IL-12 is one of the most potent factors in the induction of a cellular or T_{H}1 response (Manetti 1993). A T_{H}1 response results in the development of cells that produce high amounts of IFNγ (Moser and Murphy 2000). The T_{H}1 cell production of cytokines, including IFNγ, lead to a cellular response that ultimately ends in the destruction of intracellular pathogens (Mosmann et al. 1986, Sher and Coffman 1992). Maturation of DCs in the absence of IL-12 has been shown to induce a T_{H}2 response (Moser and Murphy 2000). A T_{H}1 type response has also been effective in experimental anti-tumor immune responses (Mosmann T 1996, Trincheri 1994, Brunner et al 2000).

DCs have been used extensively for vaccination against a variety of protein antigens and DC vaccines have also been found to be powerful stimulators of the cellular immune response to tumors (reviewed in Banchereau et al. 2000). DCs act as potent initiators of tumor immunity in murine tumor models. The development of tumors in experimental animals can be induced by injection of established tumor cell lines derived from a number of different tissues (Brunner et al. 2000). DCs pulsed with tumor antigen or cell lysate are effective anti-tumor vaccines against subsequent tumor cell challenge. A number of tumor antigen pulsing methods have met with success in experimental settings. Co-culture (Celluzi 1998) or fusion of DCs with whole tumor cells (Gong 1997) provides protection from subsequent challenge with viable tumor cells. Prophylactic benefit was also seen using DCs pulsed with known tumor antigens in peptide or whole protein form (Zitvogel 1996, Paglia 1996) and with DCs transfected with a known tumor antigen (Song et al. 1997, Kaplan et al. 1999). There have been reports of therapeutic efficacy of DC-based tumor vaccines as well. Rejection of established tumors and lung metastases has been seen using DC-tumor fusion (Gong 1997), or DC-tumor co-culture (Celluzi 1998), in addition tumor peptide or lysate pulsed DCs (Mayordomo 1996, Labeur 1999) have also shown some therapeutic benefit in murine models.

The results seen in experimental murine systems have led to the initiation of tumor antigen presenting DC clinical trials in human patients. DC based therapy requires that the DCs be exposed to tumor antigen(s) that are associated with the tumor. Clinical trials are now underway in many malignancies including B cell lymphoma, melanoma and prostate cancer. In each of these clinical trials a known antigen is being used to treat established disease. The availability of a known antigen allows for monitoring of the immune response to the antigen that is given to the DC. These studies have shown the presence of an immune response to the antigen that was absent prior to initiation of the DC treatment (Fong and Engelman 2000). However most studies do not attain a high cure rate with these treatments.

A number of studies in experimental animals have been undertaken to improve the anti-tumor immune response against a known tumor antigen. Many such studies aim at increasing the immune response to a given antigen through the use of DC vaccines or therapies. A number of different tumor associated or tumor specific Ag have been used. One such tumor model uses a well-established tumor cell line, CT26. CT26 is an N-nitroso-N-methylurethane induced BALB/c undifferentiated colon carcinoma. This tumor grows progressively in animals after subcutaneous or intra-venous injection (Wang et al. 1995). The transfection of this tumor with the bacterial lac-Z gene leads to the expression of β-galactosidase in the tumor cells. This variant of CT26, that is called CT26.CL25 has been established as a progressively growing tumor (Wang et al. 1995). In this system, β-galactosidase acts as a surrogate tumor antigen.

β-Galactosidase (β-gal) is an enzyme that cleaves substrates including lactose and o-nitrophenyl-62 -D-galactopyranoside (ONPG). β-Gal has been extensively studied in investigations into the E. coli lactose operon and it has also been used as a marker for measuring the efficiency of gene transfer. As a result of these past studies, many assays have been developed to detect and quantify the presence and activity of β-gal. The ease of detection of β-gal has made it a popular choice for immunological investigations.

A number of investigators have used β-gal loaded or β-gal transfected DCs as an anti-tumor vaccine or treatment for CT26.CL25 tumors or other tumor models that employ β-gal expression, i.e. the P815 β-gal transfectant P13.4 (Paglia 1996, Song 1997, Specht 1997). While the study by Paglia et al. showed that soluble β-galactosidase protein loading of DCs could be reasonably effective in evoking an immune response, there was room for improvement. One drawback to the pulsing of DCs with a soluble antigen was that a fairly high concentration of antigen was needed for the DC pulse in order to provide protective immunity to a tumor challenge. To be useful clinically a novel vaccine strategy using DCs should be technically feasible and be applicable to a large number of different tumor types. Targeting of the antigen to surface receptors expressed on the DC offers a way to improve antigen uptake and lower the amount of antigen required to elicit immunity.

Previous studies have shown the benefit of targeting of antigen to surface receptors on DCs. Antigen-antibody complexes (Fanger 1996), Ag-Ig fusion proteins (You et al. 2001) and heat shock protein-peptide constructs (Suzue K 1997, Arnold-Schild 1999, Todryk 1999) have been shown to increase antigen binding and delivery to DCs and enhance the immunogenicity of the bound antigen. Improved immunogenicity of targeted antigen has also been seen in similar studies using other, non-specific targeting methods such as cationic liposome association with Ag (Ignatius 2000), production of apoptotic bodies from tumor cells (Rubartelli 1997, Albert 1998a, Albert 1998b), and more recently cationic fusogenic peptides (Laus 2000). Each of the referenced techniques are associated with either significant technical expertise (cationic liposome association and cationic fusogenic peptides) or labor intensive isolation of antigen and cell culture techniques (purification of apoptotic bodies). These methods may also require construction and expression of fusion proteins, the availability of a specific antibody-antigen pair, complex manipulations or a large amount of tumor material to enhance targeting to antigen presenting cells. These drawbacks may limit the utility of such techniques.

While DC based immunotherapies have met with some success, to date these therapies have had limited clinical applicability. Improving on the methods of Ag delivery to DCs may further the clinical applicability of DC vaccination strategies by (1) reducing the amount of Ag needed to efficiently initiate an immune response and (2) potentially allowing for increased MHC class I loading of peptides from exogenous proteins thus achieving greater priming of cytotoxic T cell responses. Whereas previous studies have shown benefit in targeting antigen to APC surface receptors, such approaches are limited by their dependence on either significant technical expertise or labor-intensive isolation and cell culture techniques (Fanger et al. 1996, Suzue et al. 1997, You et al. 2001). Accordingly, there is a need for development of simple methods to enhance the immunogenicity of targeted antigens.

### SUMMARY OF THE INVENTION

The present invention provides compositions and methods for enhancing the immunogenicity of antigens. The method comprises covalently linking (also referred to as covalent coupling) the antigen to proteins or glycoproteins on the surface of dendritic cells and using the dendritic cells to elicit an immune response. The mild biochemical modification employed by this approach minimizes denaturation of the Ag. In addition, the viability of cells is preserved.

In one embodiment, a model tumor antigen, β-galactosidase (β-gal) was covalently coupled to proteins or glycoproteins on the surface of DCs. DCs with covalently linked Ag on their surface were compared to DCs pulsed with soluble Ag for the ability to generate a tumor specific immune response in mice. Covalently linked β-gal-DCs proved to be superior to soluble β-gal loaded DCs in generating both protective and therapeutic anti-tumor immunity.

This technique can be used with a wide range of antigens such as proteins or peptide fragments of known tumor or microbial proteins or tumor cell and bacterial lysates that contain a variety of antigenic components.

The invention also provides compositions for eliciting an immune response. The composition comprises dendritic ells having one or more antigens covalently linked to the surface molecules, preferably proteins or glycoproteins.

The invention also provides a method for making a composition for eliciting an immune response in an individual. The method comprises obtaining dendritic cells from the individual (or a syngeneic source), covalently coupling an antigen to one or more proteins or glycoproteins on the surface of the dendritic cells; and reinfusing the covalently coupled dendritic cells into the individual.

The invention also provides a method of reducing the growth of a tumor by obtaining a tissue sample from the tumor; isolating an antigen from the tissue sample or preparing cell lysates; obtaining dendritic cells from the individual; covalently linking the purified, partially purified or unpurified antigen to one or more surface proteins on the dendritic cells; and reinfusing the covalently linked dendritic cells into the individual.

The invention also provides a method for reducing the recurrence of the growth of tumors in an individual in which the tumor has been surgically removed by obtaining a tissue sample from the tumor which has been surgically removed; isolating an antigen from the tissue sample or preparing a cell lysate from the tumor; obtaining dendritic cells from the individual; covalently linking the antigen or the cell lysate to one or more surface proteins or glycoproteins on the dendritic cells; and reinfusing the covalently linked dendritic cells into the individual.

The invention also provides a method for reducing the incidence of occurrence of tumors by identifying an antigen known to be present in the tumors, obtaining dendritic cells from an individual; covalently linking the antigen from the tumor to one or more surface proteins on the dendritic cells; and reinfusing the covalently linked dendritic cells into the individual.

List of Abbreviations Used

The following abbreviations are used throughout the application:

Ab, Antibody

Ag, Antigen

APC, Antigen Presenting Cell

β-gal, beta-galactosidase

BSA, Bovine Serum Albumin

CDR, Complemetarity Determining Region

CCR_, CC type chemokine receptor_{—}

CpG, Cytidine-phosphate-Guanosine

Con A, Concanavalin A

CTL, Cytotoxic T Lymphocyte

DC, Dendritic Cell

DC1, human myeloid DC subset

DC2, human lymphoid DC subset

DMEM, Dulbecco's Modified Eagle Medium

EDTA, Ethylenediaminetetraacetic acid

ELISA, Enzyme linked immunosorbent assay

ELISPOT, Enzyme linked immunosorbent spot (assay)

Fc, Crystalizable Fraction of the immunoglobulin molecule

FCS, Fetal Calf Serum

IFNγ, Interferon gamma

Ig, Immunoglobulin

IL-_, Interleukin-_{—}

ip, intraperitoneal

iv, intravenous

LPS, Lipopolysaccharide

MESNa, 2-Mercaptoethanesulfonic acid Sodium salt

MHC, Major Histocompatability Complex

MLR, Mixed Lymphocyte Reaction or Mixed Leukocyte Reaction

MTT, (3-(4,5-dimethylthiazol-2-yl)-2,5-diphenyl tetrazolium bromide)

NK, Natural Killer cells

NKT, Natural Killer-T cells

ONPG, o-nitrophenyl-β-D-galactopyranoside

OVA, Chicken ovalbumin

PBL, Peripheral Blood Leukocyte

PBS, Phosphate Buffered Saline

PDTP, 3-(2-pyridyldithio)propionyl

PSA, Prostate Specific Antigen

RANK, Receptor Activator of Nuclear factor-kappaB

rmGM-CSF, recombinant mouse Granulocye, Macrophage Colony Stimulating Factor

RPMI, Roswell Park Memorial Institute

SAC, Staph. aureus strain Cowan I cells

sc, subcutaneous

SCID, Severeve Combined Immunodeficient (mouse)

SLC, Secondary lymphoid tissue chemokine

SPDP, N-succinimidyl 3-(2-pyridyldithio) propionate

T_{H}1, T cell helper subset 1 (cellular immune response)

T_{H}2, T cell helper subset 2 (humoral immune response)

TNFα, Tumor Necrosis Factor-alpha

TRANCE, tumor necrosis factor [TNF]-related activation-induced cytokine

TRIS, Tris(hydroxymethyl) aminomethane

V_{H}, Variable region of the IG heavy chain

### BRIEF DESCRIPTION OF THE DRAWINGS

FIG. 1. Day 10 bone marrow culture derived cells exhibit dendritic cell morphology as exemplified by extensive cytoplasmic “veils” in non-adherent cells and stellate projections in attached cells.

FIG. 2. Flow cytometric analysis of day 7 GM-CSF cultured bone marrow dendritic cell phenotype. (a) Cell granularity and size are consistent with the DC phenotype, (b) and (c) The open histogram represents the level of staining obtained with an isotype control Ab, and the filled histograms represent the level of staining with the Ab indicated CD11c (b), MHC class II molecules (c).

FIG. 3. Flow cytometric investigation of DC maturation. Day 10 BM cultured cells were assayed for MHC class II (y axis) and CD86 (x axis) expression 16 hours after transfer to a 6 well tissue culture plate. The cells were incubated overnight with media +GMCSF only (a) or with the addition of 100 ng/ml LPS (b), 1 μg/ml CpG 1826 (c), or 6 μg/ml CpG 1826 (d).

FIG. 4. Stimulation of the MLR by GM-CSF cultured BM cells. 5×10⁵ Allogeneic T cells were mixed with varying doses of day 9 GM-CSF cultured BM cells (▪) or splenoctyes (□). After a three-day incubation period, cell number/activity was determined by an MTT assay. Each data point represents three separate wells. The error bars are demonstrative of one standard deviation.

FIG. 5. IL-12 production by GM-CSF BM cells in response to CpG 1826. Day 9 GM-CSF cultured bone marrow cells were transferred to a tissue culture treated well at 5×10⁵ cells in 0.5 ml complete medium per well. To each well was added 0.5 ml of media with no additional factors (None) or a maturation factor: LPS (100 ng/ml final conc.), or CpG 1826 at a final concentration of 1, 6, or 12 μg/ml. Medium supernatant was collected and assayed 20 h later. Each column is representative of triplicate samples from duplicate plates. The error bars are representative of one standard deviation.

FIG. 6. Titration of the optimal dose of CpG for the induction of IL-12 by BM cultured cells. Day 9 GM-CSF cultured bone marrow cells were transferred to a tissue culture treated well at 5×10⁵ cells in 0.5 ml complete medium per well. To each well was added 0.5 ml of media with a titrated dose of CpG 1826 at final concentrations as labeled. Medium supernatant was collected and assayed 20 h later. Each column is representative of triplicate readings of duplicate wells. The error bars are representative of one standard deviation.

FIG. 7. Coupling of a protein antigen to proteins on the surface of DCs through the use of SPDP. (a) Introduction of a 3-(2-pyridyldithio) propionyl (PDTP) groups into a protein by aminolysis. (b) Reaction between the modified protein Ag containing PDTP groups and the DC through thiol-disulfide exchange to form the disulfide linked protein-DC conjugate.

FIG. 8. PDTP-β-Gal binding to the surface of DCs. Either soluble β-Gal (open bar) or PDTP modified β-Gal (colored bar) was added to a titrated number of DCs. The cells were washed extensively after a 1-hour incubation. Cell surface β-Gal activity was determined by an ELISA to detect the cleavage of ONPG. Each column is representative of triplicate wells. The error bars are representative of one standard deviation. This experiment was repeated 3 times with similar results.

FIG. 9. Loss of β-Gal activity through the use of MESNa. 1×10⁶ DCs were incubated with PDTP-β-Gal for 60 min. followed by treatment with MESNa (open bar) or PBS (filled bar). The cells were washed extensively after a 1-hour incubation and β-Gal activity was determined using an ELISA. Each column is representative of triplicate wells. The error bars are demonstrative of one standard deviation. This experiment was repeated twice with similar results.

FIG. 10. Internalization of covalently coupled surface β-Gal by DCs. Initially, 1.2×10⁷ DCs were incubated with PDTP-β-Gal for 60 min. DCs were incubated at 37° C. for the indicated amount of time. At the end of the incubation period the DC were treated with MESNa. Intact (open bar) or lysed (closed bar) cells were then assayed for β-Gal activity using an ELISA with 1×10⁶ DCs per well. Each column is representative of triplicate wells. The error bars are one standard deviation. This graph is representative of three experiments.

FIG. 11. Ag pulsed DCs stimulate the allogeneic MLR. Escalating doses of unpulsed DC (♦), soluble β-Gal (□) and PDTP-β-Gal (●) were added to 2×10⁵ allogeneic lymphocytes. After a three-day incubation period, cell number/activity was determined by an MTT assay. Each data point represents three separate wells. The error bars are demonstrative of one standard deviation from the mean.

FIG. 12. Survival of DCs vaccinated mice after CT26.CL25 challenge. Groups of mice were vaccinated with 5×10⁵ cells; unpulsed DC (◯), soluble β-Gal pulsed DCs (▪), PDTP-β-Gal pulsed DCs (⋄) or left unvaccinated (▴). Mice were challenged subcutaneously with 5×10⁵ CT26.WT tumor cells 17 to 49 days after treatment. Tumor growth was monitored weekly and survival ended at sacrifice, when one dimension of the tumor exceeded 2 cm. The data from 6 experiments were combined to produce this figure.

FIG. 13. Growth of CT.WT tumors in DC Vaccinated Mice. Groups of mice were vaccinated with 5×10⁵ cells; unpulsed DCs (▪), soluble β-Gal pulsed DCs (▴), PDTP-β-Gal pulsed DCs (●) or left unvaccinated (♦). Twenty days later the mice were subcutaneously challenged with 5×10⁵ CT26.WT tumor cells. Each data point represents the average tumor size in five mice. Tumor growth was monitored weekly. Tumor volume was calculated as described. Mice were sacrificed when one dimension of their tumor exceeded 2 cm. This preparation of DCs (PDTP-beta-Gal pulsed) was able to protect mice against challenge with CT26.CL25 (□). There was no significant difference in tumor growth between any of the cell lines that were challenged with CT26.WT (p≧0.5).

FIG. 14. Survival of mice challenged with CT26.CL25 7 weeks after DC vaccination. Groups of mice were vaccinated with 5×10⁵ cells; unpulsed DCs (▪), soluble β-Gal pulsed DCs (▴), or PDTP-β-Gal pulsed DCs (●). Forty-nine days later the mice were subcutaneously challenged with 5×10⁵ CT26.CL25 tumor cells. Tumor growth was monitored weekly and survival ended at sacrifice, when one dimension of the tumor exceeded 2 cm. The data from 2 experiments were combined to produce this figure.

FIG. 15A-C. DC based treatment of established tumors. Mice were injected S.C. with 5×10⁵ CT26.CL25. Ten days later the mice were treated with a contra lateral S.C. DC injection as indicated. Each group contained 5 mice and each mouse is represented individually. Tumor growth was monitored weekly. Tumor volume was calculated as described. Mice were sacrificed when one dimension of their tumor exceeded 2 cm.

FIG. 16. The addition of CpG to in vitro DC cultures results in an increase in antigen specific IFNγ producing cells. Mice were vaccinated as indicated on the X-axis. Twelve days later splenocytes from the vaccinated animals were isolated and restimulated for 6 days with β-gal positive P13.4 tumor cells. The cells were recovered and incubated overnight in an ELISPOT plate in the presence of P13.4 cells. The responder cells were titrated at 2×10⁵ (gray column), 1×10⁵ (white column) and 5×10⁴ (black column) per well. The plates were used in an ELISPOT assay to detect IFNγ producing cells. Each column is the average of triplicate wells. The error bars are one standard deviation from the mean. This experiment was repeated and yielded comparable results.

FIG. 17. IFNγ producing cells obtained from β-gal-pulsed and β-gal-conjugated vaccination. Mice were vaccinated as indicated on the X-axis. Splenocytes from the vaccinated animals were isolated and restimulated with irradiated P13.4 cells. Five days later the cells were recovered and incubated overnight in an ELISPOT plate in the presence of β-gal positive P13.4 tumor cells. The responder cells were assayed at 2×10⁵ (gray column), and 1×10⁵ (white column). The plates were used in an ELISPOT assay to detect IFNγ producing cells. Each column is the average of quadruplicate wells. The error bars are one standard deviation from the mean. This figure is representative of 3 independent experiments.

FIG. 18. IFNγ production by CD8⁺ and CD8⁻ cells. Mice were vaccinated as indicated on the X-axis. Splenocytes from the vaccinated animals were isolated and restimulated with irradiated P13.4 tumor cells for 5 days. The recovered cells were separated into CD8⁺ and CD8⁻ cells and then were incubated overnight in an ELISPOT plate in the presence of P13.4 cells. The responder cells were plated at 5×10⁴ CD8⁺ cells (gray column), and 5×10⁴ CD8⁻ cells (white column) in an ELISPOT assay to detect IFNγ producing cells. Each column is the average of quadruplicate wells. The error bars are one standard deviation from the mean. This figure is representative of 2 independent experiments.

FIG. 19. Requirement of β-gal expressing cell line for in vitro restimulation period. Mice were vaccinated as indicated on the X-axis. Splenocytes from the vaccinated animals were isolated and restimulated for 6 days with either β-gal negative CT26.WT cells (open columns) or with the β-gal expressing CT26.CL25 tumor cell line (filled columns). The recovered cells were then were incubated overnight in an ELISPOT plate in the presence of CT26.CL25 cells. The responder cells were plated in 3 wells and the average values are reported. The error bars are one standard deviation. This experiment was repeated twice at lower responder to stimulator cell ratios with similar results.

### DETAILED DESCRIPTION OF THE INVENTION

The present invention provides a method for enhancing the immunogenicity of antigens by covalently linking them to the proteins or glycoproteins on the surface of dendritic cells. Covalent linkage can be achieved by methods well known in the art. Once the antigen has been covalently linked to the DC surface, the DCs can then be used to elicit an immune response. As an illustration, dendridic cells from the bone marrow have been used.

The method of the present invention can be used with any antigen and can be used for prophylactic as well as therapeutic purposes. Examples of antigens include but are not limited to tumor related antigens such as Inmunoglobulin idiotypes, Mage, BAGE, MART, SV40T antigen, EBNA-1, Her-2/neu, Bcr/Ab1, Ras, Tyrosinase, Alpha-fetoprotein, Prostate specific antigen; viral antigens such as viral coat proteins and viral capsid proteins; and bacterial antigens such as bacterial coat proteins and bacterial products such as heat killed toxins (e.g., tetanus toxoids) etc.

Antigens useful for the invention can be obtained commercially or prepared by standard methods. For example, tumor antigens can be obtained by preparation of tumor cell lysates which are prepared by repeatedly freezing and thawing tumor cells/tissues in phosphate buffered saline containing leupeptin and aprotinin (obtained from either fresh tumor biopsy tissues or from tumor cells generated in vitro by tissue culture). The freezing and thawing results in the lysis of cells. The tumor lysate is obtained by centrifugation and harvesting the supernatant fluid. The tumor cell lysates can be used immediately or frozen and stored at −70° C. until ready for use. The cell lysate can itself be used for covalent coupling to DCs. The antigen can be used in a purified form or in partially purified or unpurified form as cell lysate.

The experiments described here demonstrate that covalent linkage of Ag to the surface proteins or glycoproteins of DCs enhances the immune response elicited to that Ag. The process of covalently coupling Ag to DCs is simple and mild enough that DC viability is well preserved. While not intending to be bound by any particular theory, it is considered that covalently linked antigen is internalized, processed and presented by DCs. This is evidenced by the vaccination of mice with PDTP-β-gal pulsed DCs and the subsequent recovery of CD8⁺ T cells that respond specifically to β-gal expressing tumor cells. The processing and presentation of β-gal was also seen in vivo where immunized mice were protected from a challenge with a β-gal expressing tumor yet were not protected from challenge with the parental non-β-gal expressing tumor. When compared to pulsing DCs with soluble Ag, covalent coupling of β-gal to proteins on the surface of DCs increased the number of mice protected against a β-gal expressing tumor when the DCs are used as a vaccine. Covalent coupling of β-gal to proteins on the surface of DCs is also shown to allow the DCs to be used as an effective treatment for established tumors where soluble Ag pulsed DCs have failed. The rapidity of the response to PDTP-β-gal loaded DCs suggests the presence and augmentation of an innate response that is not seen with soluble β-gal loaded DCs. The immunological benefit of covalent coupling of β-gal to DC surface proteins has been observed in both vaccination against and therapy of a β-gal expressing tumor. The results of these in vivo protective and therapeutic studies suggest that both the adaptive and innate arms of an anti-tumor immune response are active in controlling the growth of a subcutaneous tumor.

Covalent coupling of Ag to proteins or glycoproteins on the DC cell surface does not require complex biochemical or molecular manipulation. Covalent coupling of antigen to proteins on the surface of DCs requires an initial mild modification of the antigen, followed by interaction with the DCs to effect a covalent association with the target cell. The reactions required for this procedure are performed under gentle physiological conditions, thereby minimizing denaturation of the antigen and preserving cell viability.

Covalent coupling of antigens to the surface proteins or glycoproteins of dendritic cells can be accomplished by well known methods that are within the purview of those skilled in the art. A wide variety of compounds including homobifunctional and heterobifunctional reagents are available for covalent coupling.

One class of these compounds, heterobifunctional reagents, allows for the covalent linkage of two proteins to each other through the use of two different reactive groups. Any reactive group can be used. An example of such a bifuctional reagent is N-succinimidyl 3-(2-pyridyldithio)propionate (SPDP). SPDP is a heterobifunctional reagent that works to covalently link together two proteins through the creation of a disulfide bond. When a heterobifunctional reagent such as SPDP is used, coupling of the reagent to the first protein and linking it to the second protein can be carried out in separate sequential steps because the two reactive groups of the SPDP are directed toward different functional groups on the proteins. While not intending to be bound by any particular theory, it is considered that SPDP acts primarily in the following way. SPDP contains one N-hydroxysuccinimide ester moiety and one 2-pyridyl disulfide moiety. The first reaction occurs when the hydroxysuccinimide esters react with the amino groups on the protein Ag, this reaction gives rise to stable amide bonds. Treatment of the protein Ag with iodoacetamide prior to reaction with SPDP blocks any free thiol groups that would react with the 2-pyridyldithio moiety of the SPDP molecule and allows the reaction to occur without intramolecular crosslinking or homoconjugation of the protein Ag. The protein Ag now contains 3-(2-pyridyldithio)propionyl (PDTP) groups that are able to react with a second protein. When the PDTP-Ag is added to a second protein (or to cell surface proteins) the second reaction proceeds. The 2-pyridyl disulfide groups then react with free thiol groups to form disulfide bonds. Those skilled in the art will recognize that other heterobifunctional covalent linkers can also be used.

The coupling reaction is preferably mild for use in antigen conjugation to the surface of a cell so that cell viability and function is preserved. When using this method to covalently link Ag to the surface of a DC the ability of that cell to function as a competent antigen-presenting cell is preserved. The ability of SPDP to function under mild physiological conditions allows for the coupling of a protein antigen to the surface of DCs with no significant reduction in cell viability.

Those skilled in the art will recognize that other heterobifunctional reagents including but not limited to SMPB, SIAB, SMCC, SMPH and SMPT can also be used. Examples of other heterobifunctional reagents can be found in U.S. Pat. Nos. 4,529,712 and 4,232,119 (incorporated herein by reference). Further, several such reagents are listed in commercial catalogs e.g., Pierce Chemical Co. catalog.

The advantages of the present method are that DCs coupled to Ag and administered as a vaccine or therapy according to the present invention elicit an enhanced immune response compared to the immune response generated by DCs loaded with soluble Ag. Furthermore covalent linkage of antigen to cell surface proteins of in vitro culture derived DCs will allow for the immunological benefits of receptor mediated Ag loading of DCs without the excessive technological limitations associated with the previous approaches.

In one embodiment are provided dendritic cells in which one or more antigens have been covalently linked to surface molecules as described herein. The DCs before and after covalent coupling to Ag, can be used fresh or stored frozen. For freezing of dendritic cells, standard method known to those skilled in cell culture techniques can be used. For example, 5×10⁶ cells/ml in RPMI tissue culture medium containing fetal calf serum (10%) and DMSO (10%) are frozen using a controlled freezing apparatus and stored in liquid nitrogen until they are to be used.

The DCs may also be induced to mature in culture. Several factors are known for maturation of DCs including exposure to GM-CSF, LPS or CpG oligonucleotides, or crosslinking of CD40. As used herein CpG olignucleotide means an oligonucleotide containing at least one unmethylated CpG dinucleotide. One examples are such CpG oligonucleotide is as follows:

TCCATGACGTTCCTGACGTT (SEQ ID NO:1). Other examples can be found in U.S. Pat. Nos. 6,406,705 and 6,239,116 and in Chu et al., 1997. As described in the example given herein and as known in the art, maturation can be induced in the DCs.

In another embodiment is provided a method for making a composition for use in eliciting an immune response. The method comprises the step of obtaining dendritic cells from an individual (or identical twin i.e., syngeneic) and covalently linking the antigen to the surface molecules of the dendritic cells such that the immunogenicity of the antigen is increased over when the Ag is not covalently coupled to the DCs.

In another embodiment is provided a method for increasing the immunogenicity of an antigen. The method comprises the steps of obtaining dendritic cells from an individual in need of treatment, covalently linking an antigen to surface protein and glycoprotein molecules of the dendritic cells and reinfusing the dendritic cells into the individual to elicit an immune response. While it is preferable to use dendritic cells of the recipient, dendritic cells from an identical twin (syngeneic) can also be used.

The present invention can be used for preventive as well as prophylactic purposes. The following examples are provided to illustrate the present invention and are not meant in any way to be restrictive.

Materials and Methods

Materials/Reagents

N-Succinimydyl-3(2-pyridyldithio)propionate (SPDP) (Sigma, St Louis, Mo.) was prepared at 20 mM in absolute ethanol and used as outlined below. 2-mercaptoethanesulfonic acid - sodium salt (MESNa) (Sigma, St Louis, Mo.) is a membrane impermeant reducing agent that was prepared to 10 mM in 50 mM Tris, pH 8.6, 100 mM NaCl, 1 mM EDTA, and 0.2% BSA and used as outlined below. Iodoacetamide (Sigma, St Louis, Mo.) was used at 0.5 M or 1 M in 0.3 M Tris buffer as detailed below. Concanavalin A (Con A) (Sigma, St Louis, Mo.) is a tetrameric protein with carbohydrate binding specificity (lectin) that has the ability to induce mitogenic activity in T lymphocytes and to increase the synthesis of cellular products. Con A was used in the ELISPOT assay to provide a positive control for IFNγ production as described below. Lipopolysaccharide (LPS) (Sigma, St Louis, Mo.) was derived from Escherichia. coli (Serotype 055:B5) and was used as described below. The synthetic oligodeoxynucleotide CpG 1826 was produced by the Biopolymer facility at Roswell Park Cancer Institute and was phosphorothioate-modified to decrease its susceptibility to phosphodiesterase degradation. The sequence of CpG 1826 was obtained from a previous report (Chu et al. 1997). MTT (3-(4,5-dimethylthiazol-2-yl)-2,5-diphenyl tetrazolium bromide) (Sigma, St Louis, Mo.) was used as a measure of T cell proliferation.

Experimental Animals

The animals used to isolate DCs were BALB/c mice (Taconic, Germantown, N.Y.) of 8 to 12 weeks of age. In each experiment the animals were age and sex, matched. C57BL/6 mice (Taconic, Germantown, N.Y.) were used only as donors for splenocytes that were used in the mixed leukocyte reactions (MLR). C57BL/6 mice (Taconic, Germantown, N.Y.) were also from 8 to 12 weeks of age and were used as a source of responder cells in mixed leukocyte reactions.

Cell Lines

CT26 is an N-nitroso-N-methylurethane induced BALB/c (H-2^{d}) undifferentiated colon carcinoma. This tumor grows progressively in BALB/c mice after subcutaneous or intra-venous injection (Wang et al., 1995). The transfection of this tumor with the bacterial lac-Z gene leads to the expression of β-galactosidase in the tumor cells (Wang et al. 1995). This variant of CT26, CT26.CL25 has been established as a progressively growing tumor. CT26.WT and CT26.CL25 were obtained from Dr. Nicholas Restifo (Surgery Branch in the Division of Clinical Sciences, NCI). The P815 cell line is a mouse mastocytoma line of DBA/2 origin (H2^{d}). The P815 cell line and the P13.4 cell line that is a beta-galactosidase (beta-gal) expressing P815 subclone were both obtained from Dr. Michael Bevan (University of Washington). CT26.WT and CT26.CL25 were maintained in complete medium; RPMI 1640 (Gibco-BRL, Grand Island N.Y.) supplemented with Penicillin (20 U/ml, Gibco-BRL, Grand Island N.Y.), Streptomycin (20 μg/ml, Gibco-BRL, Grand Island N.Y.), L-glutamine (2 mM, Gibco-BRL, Grand Island N.Y.), 2-mercaptoethanol (50 μM, Sigma, St Louis, Mo.) and 10% heat inactivated and filtered FCS (Gibco-BRL, Grand Island N.Y.). The P815 and P13.4 cell lines were maintained in sterile filtered Dulbecco's Modified Eagle Medium/F12 nutrient mixture with the addition of 10% heat inactivated FCS (Gibco-BRL, Grand Island, N.Y.).

Murine DC Culture

Murine bone marrow derived dendritic cells were generated by the method described by Lutz et al. To prepare mouse bone marrow the femurs and tibiae from the desired number of mice were extracted and the surrounding tissue was removed by rubbing with gauze squares. The bones were soaked in 70% ethanol for 2-5 minutes and were then washed with PBS. Both ends of the bones were cut with scissors and the marrow was flushed out with PBS using a 0.45 mm syringe. Clusters within the suspension were dissociated by vigorous pipetting and the cells were washed once in PBS. At this point (day 0) the bone marrow derived leukocytes were plated in 100 mm bacteriological petri dishes at 2×10⁶ cells per dish in 10 ml of complete medium supplemented with 200 U/ml rmGM-CSF (Peprotech, Rocky Hill, N.J.). At day 3, 10 ml of complete medium supplemented with 200 U/ml rmGM-CSF was added to each of the plates. On day 6 and 8, 10 ml of the medium was removed from each plate and was replaced with fresh medium plus 200 U/ml rmGM-CSF. DCs were harvested on day 9 for antigen pulse prior to vaccination.

FACS Staining

DCs were collected, counted and their viability was determined by trypan blue exclusion. The cells were subjected to centrifugation at about 250×g for 3 minutes at 4° C. in quench solution (PBS with 1% bovine serum albumin and 0.5% normal rat serum). The cells were placed into 4 ml culture tubes (Becton Dickinson, Lincoln Park N.J.) at 5×10⁵ cells per tube, with one tube from each group of cells being set aside as an auto-fluorescence control. The cells were once again subjected to centrifugation at 1500 rpm for 3 minutes at 4° C. in 3ml of quench solution. Following the wash the supernatant was decanted and 10 μl blocking immunoglobulin (1 mg/ml) was added to each tube with the tubes then being incubated on ice for 10 minutes. Following this incubation, the specific primary labeled, antibodies (Abs) were added at concentrations consistent with the manufacturers instructions. The specific Abs were incubated on ice for 15 minutes. The cells were washed by the addition of 3 ml of quench solution for each tube followed by centrifugation at about 250×g for 3 minutes at 4° C. After this final wash the cells were fixed by the addition of 500 μl of 3% formalin. The tubes were stored in the dark at 4° C. until they were analyzed by flow cytometry. The antibodies used to stain the DCs were; anti-CD11c (HL3, Armenian hamster IgG, group1,λ), anti-CD86 (GL 1, rat IgG₂ₐ, κ), anti-Ly6c (AL-21, rat IgM, κ), and anti-I-A^{d} (M5/114.15.2, rat IgG_{2b}, κ) (BD PharMingen San Diego Calif.).

MLR/MTT Assay

Spleens were collected from naïve C57BL/6 or BALB/c mice. The spleens were disrupted by maceration between two autoclaved frosted microscope slides. The cells were suspended in cold PBS and subjected to centrifugal force (1000 rpm for 7 min.). The cell pellets were then resuspended in ice cold 0.83% ammonium chloride for five minutes. The cells were then spun down (1000 rpm for 7 min.) and washed by resuspending in complete RPMI 1640 medium followed by another centrifugation (1000 rpm for 7 min.). The BALB/c splenocytes were irradiated and used as stimulator cells in the MLR. The C57BL/6 mouse splenocytes were placed into tissue culture flasks at 2×10⁷ cells per flask. After a 1-hour incubation the non-adherent cells were removed and used as responders in the one-way MLR. BALB/c DCs were cultured as described above. The cells were either left unpulsed or pulsed with soluble β-galactosidase or PDTP-β-galactosidase. The DCs were irradiated (5000 rad) and then plated in 96 well plates at 2.5×10⁴ to 5×10⁵ cells per well. When BALB/c splenocytes were used as stimulator cells they were irradiated (5000 rad) and then plated at 1×10⁵ to 5×10⁶ cells per well. The responder cells were added at 1×10⁵ cells per well in a 96 well plate. Control wells were included that contained stimulator cells alone, responder cells alone or medium alone. Each mixture was plated in triplicate wells.

After a 72-hour incubation cell proliferation was measured using a colorimetric, MTT (3-(4,5-dimethylthiazol-2-yl)-2,5-diphenyl tetrazolium bromide) assay that has been described previously (Mossman 1993). Briefly, 100 μl of medium was removed from each well and 10 μl of stock MTT solution (5 mg/ml in phosphate buffered saline) was added to each well. The plates were incubated at 37° C. for 4 hours. The MTT is reduced to a blue insoluble formazan product by living cells. Two hundred microliters of acid isopropanol (0.04 N HCl in isopropanol) was added to all of the wells and mixed thoroughly to completely dissolve the dark blue formazan crystals. The supernatant fluid was transferred to an ELISA plate that was read on an ELISA reader at a single wavelength of 540 nm.

Antigen Pulse of Murine DCs

DCs were prepared from mouse bone marrow as described above. The DCs were grown in complete medium. The DCs were harvested on day 9 and washed extensively (4×) in PBS to remove residual medium related protein. The cells were split into three groups for the antigen pulse. Group 1 DCs were pulsed with PBS alone, group 2 DCs were pulsed with soluble β-galactosidase at 1 mg/ml PBS for 30 minutes and then 100 g/ml complete medium for 16 hours and group 3 DCs were pulsed with PDT-β-galactosidase at 1 mg/ml for 30 minutes and then 100 μg/ml for 16 hours. Each group of cells was initially plated in 2 wells of a 6 well plate at 1×10⁷/well/ in 500 μl PBS or Ag in PBS for 30 minutes. Following the first stage of the Ag pulse 4.5 ml of complete medium, including GM-CSF, was added to each well and the DCs were incubated overnight. At the end of the incubation period 2 ml of medium was removed from each well and replaced with 3 ml of complete medium +600U GM-CSF and 6 μg of 1826 CpG. The cells were then incubated for an additional 6 hours. At the end of the final incubation the DCs were collected and washed 4 times in PBS to remove any residual antigen, FCS or CpG. The DCs were counted and injected as indicated in the results section.

Preparation of PDTP-proteins

To estimate the quantity of SPDP needed for the reaction with the protein the following formula was used.

$$\text{Volume(ml) of 20 mM} \text{SPDP} \text{to be added} = \frac{Q / M W \times n \times 5}{20 \times {10}^{- 3}}$$

Where Q=quantity of protein (mg)=protein concentration (mg/ml) times the volume of protein solution (ml); MW=molecular weight of protein; n=number of amino acid groups per protein molecule to be modified. In each of the preparations an n of 5 was used.

β-Galactosidase was prepared as a 5 mg/ml solution in PBS. The protein solution was mixed with 1M iodoacetamide in 0.3 M Tris buffer with to bring the solution to 0.022 M iodoacetamide. The protein was treated for 30 minutes at room temperature with mixing and then dialyzed against 4 liters of PBS at 4° C. for 16 hours. At this point half of the protein was set aside for use as the source of soluble β-galactosidase.

The protein solution was prepared in 2 ml of PBS at a concentration of 2.5 mg/ml, 20 mM SPDP was prepared in absolute ethanol and 53.8 μl was added to the 2 ml of protein solution. The reaction was carried out for 30 minutes at room temperature with mixing. The solution was then dialyzed against 4 liters of PBS at 4° C. for at least 16 hours. Protein concentration for both β-galactosidase and PDTP-β-galactosidase was determined by optical density read at 280 nm. The preparations were diluted to 1 mg/ml of protein in PBS and sterilized using a 0.22 μM filter prior to use as antigen. The protein was then used or apportioned and frozen.

Cell Surface β-galactosidase Activity ELISA

To determine the extent of surface coupling of PDTP-β-galactosidase dendritic cells were incubated with PBS, soluble β-galactosidase in PBS or PDTP-β-galactosidase for 30 minutes at 4° C. The cells were then washed three times in PBS+5% BSA. After the final wash the cells were resuspended in PBS. An equal amount of the developing substrate containing o-nitrophenyl-β-D-galactopyranoside (ONPG) was added to 100 μl of cell suspension in PBS. The cells were incubated for 5 minutes at 37° C. in a round bottom 96 well plate. After the incubation the plate was centrifuged for 5 min at 2000 rpm at 4° C. to pellet the cells. After the centrifugation 150 μl of the supernatant was transferred to a flat bottom 96 well plate that was read at 450 nm.

Mercaptoethanesulfonic Acid-sodium Salt Resistance Assay

DCs were incubated with PDTP-β-galactosidase for 30 minutes on ice. The cells were then incubated at 37° C. in a water bath for a given length of time. At the end of each incubation period the cells were removed from the water bath and placed on ice. When all of the incubation time points were reached the cells were washed three times with PBS +5% BSA. Centrifugation was carried out at 1000 rpm for 7 minutes at 4° C. After the final supernatant was aspirated the cell pellets were resuspended in a solution comprised of 50 μl of 10 mM mercaptoethanesulfonic acid (MESNa) in 50 mM Tris, pH 8.6, 100 mM NaCl, 1 mM EDTA, and 0.2% BSA. The cells were reacted for 30 minutes at 4° C. with gentle mixing. A second amount of MESNa was added (12.5 μl of a 50 mM stock, freshly prepared before addition) and the cells were incubated at 4° C. for 30 minutes. Finally a third amount of MESNa (16 μl of a 50 mM stock) was added to the cells and the cells were incubated for an additional 30 minutes at 4°. Any excess MESNa was quenched with the addition of 25 μl of 500 mM iodoacetamide.

In vitro Restimulation (IVS)

Spleens were collected from vaccinated or naïve mice. The spleens were disrupted by maceration between two autoclaved frosted microscope slides. The cells were suspended in cold PBS and centrifuged (1000 rpm for 7 min.). The cell pellets were then resuspended in ice cold 0.83% ammonium chloride for five minutes. The cells were spun down (1000 rpm for 7 min.) and resuspended in complete RPMI 1640 medium followed by another centrifugation (1000 rpm for 7 min.). The supernatant was removed and the cells were resuspended to 2×10⁶ cells per milliliter in 5 ml complete RPMI 1640 medium. These cells were plated with 1×10⁶ tumor cells (P815, P13.4, CT26.WT or CT26.CL25) in 5 ml complete RPMI 1640 medium in a 10 cm tissue culture dish (Becton Dickinson, Franklin Lakes, N.J.) for 5 or 6 days as indicated in the results section.

CD8⁺ Cell Separation

At the end of the restimulation period the cultured cells were collected from the culture dishes and were washed by centrifugation (1000 rpm for 7 mim.). The cells were counted and portion of the cells was set aside for use in the ELISPOT assay; these cells were termed unfractionated. The second portion of the cells from each group was prepared for CD8⁺ magnetic bead enrichment using the MACS microbeads system (Miltenyi Biotech, Aubern Calif.) according to the company's protocol. These cells were washed in cold MACS buffer (PBS, 2 mM EDTA and 0.5% BSA). The cell pellet was resuspended in 90 μl of buffer per 10⁷ total cells. To this suspension was added 10 μl of MACS CD8α microbeads per 10⁷ total cells. The cells were mixed well and were incubated for 15 minutes at 6-12° C. After the incubation period the cells were washed by adding 20× the labeling volume of buffer. The cells were centrifuged at 1000 rpm for 10 minutes, the supernatant was removed completely and the pellet was resuspended in 500 μl of buffer per 10⁸ total cells. The cells were placed into a washed magnetic separation column and the negative cells were allowed to pass through and the column was washed to collect any residual cells. The column was removed from the magnetic field, was placed on a collection tube and the column was washed using 1 ml of buffer that was forced through the column using a supplied plunger. The cells were collected and washed by centrifugation (1000 rpm for 7 min.). The cells were then resuspended in complete RPMI 1640 medium and were used in the IFNγ ELISPOT assay.

IFNγ ELISPOT

Ninety-six well nitrocellulose plates (Multiscreen, Millipore, Bedford, Mass.) were coated overnight at 4° C. with 50 μl/well of 5 μg/ml anti mouse IFNγ monoclonal Ab (clone R46A2). The Ab solution was removed by inversion over a sink followed by blotting on clean paper towels. The wells were then blocked with 200 μl of blocking solution, which was 5% fetal calf serum in PBS, for 2 hours at room temperature. After the incubation the blocking solution was poured out and the plate was blotted on clean paper towels. The plate was then washed four times by submersion in PBS at room temperature. The plate was removed from submersion and left for five minutes on the bench top. The plate was carefully checked to be sure that no air was trapped in the wells during the washes. The solution was removed from the wells by pouring out and flicking over a sink followed by tapping on clean paper towels. Responder cells were recovered from the restimulation cultures and were added at varying concentration to the wells in 100 μl volumes of complete medium (RPMI 1640+10% FCS). In some experiments CD8α⁺ cells were separated from the remainder of the cultured cells (as above). Typically the cells were assayed in four replicates. Target cells, in 100 μl of complete medium were then added to the wells. The target cells can consist of P13.4 and CT26.CL25 tumor cells, CT26.WT cells, and P815 tumor cells, with or without peptide. Wells that did not receive target cells receive 100 μl of complete medium. Splenocytes from each group being tested were also plated in 100 μl of complete medium plus 100 μl of 20 μg/ml concanavalin A (Sigma, St Louis, Mo.) and serve as positive controls for the release of IFNγ. The plate was then incubated overnight at 37° C. in 5% CO₂. Following the overnight incubation, the wells were emptied as above and washed 6 times with PBS containing 0.05% Tween 20, with each wash being incubated for 3 minutes. The detection Ab was a biotinylated anti-mouse IFNγ Ab (clone XMG1.2), 0.5 μg/ml in 50 μl was added to each well. The wells were then incubated at 37° C. in 5% CO₂ for 2 hours. Following this incubation the wells were emptied and washed 6 times as described above. Avidin-horseradish peroxidase complex (Vectastain Elite Kit, Vector Scientific, Burlingame, Calif.) was prepared in PBS with 0.1% Tween 20. Each well receives 100 μl of the Vectastain solution and the plate was incubated for 1 hour at room temperature. After the incubation, the plates were washed 3 times with PBS-0.05% Tween 20 and then 3 times with PBS alone. One hundred microliters of substrate solution was added to each well of the plate, and the plate was placed in the dark, at room temperature for five minutes. The peroxidase substrate solution was prepared by first dissolving one tablet of 3-amino-9-ethylcarbazole (AEC; Sigma, St Louis, Mo.) in 2.5 ml dimethylformamide (DMF). This solution was then added to 47.5 ml of 50 mM acetate buffer. Immediately before use, 25 μl of 30% hydrogen peroxide was added and the solution was filtered to remove any particulate material that did not go into solution. The reaction was stopped by briefly washing the plates with cold tap water. The water was emptied from the plate and the plate was blotted on clean paper towels. The plastic bottom was removed from the plates, which were then left to dry overnight at room temperature. The next day the plates were counted by eye with the aid of a dissecting microscope.

Enzyme Linked Immunosorbent Assays (ELISA)

Investigations into the secretion of IL-12 by unpulsed and Ag pulsed DCs were performed using an IL-12 p70 specific Quantikine M ELISA (R&D systems, Minneapolis, Minn.). The ELISA was done according to the manufacturer's instructions. Briefly, 50 μl of assay diluent was added to each of the supplied, capture Ab pre-coated wells. Next 50 μl of standard or tissue culture supernatant sample was added per well and the plate was mixed by tapping. The plate was covered and incubated for 2 hours at room temperature (RT). The plate was washed 4 times with wash buffer and the plate was inverted and blotted between each wash. To each well was added 100 μl of anti-mouse IL-12 p70 conjugate (horseradish peroxidase labeled secondary Ab). The plate was mixed and incubated for 2 hours at room temperature. The plate was washed four more times and 100 μl of substrate solution (tetramethylbenzidine and hydrogen peroxide) was added to the wells. The plate was incubated for 30 minutes in the dark at room temperature. At the end of the final incubation step the 100 μl of stop solution was added to each well and the optical density of the individual wells was determined by reading the plates in a microplate reader set to 450 nm with a correction reading taken at 540 nm. The total concentration of IL-12 present in the supernatants was determined by plotting the OD values against a standard curve.

The Flow Cytometry department at Roswell Park Cancer institute performed a mouse cytokine array on tissue culture supernatants from DC cultures. The flow cytometer microsphere based assay (FMBA) allows for the simultaneous detection of multiple soluble cytokines in one sample tube (Reviewed in Vignali, 2000). The mouse cytokines that were tested were: IL-1 β, IL-6, and TNFα.

In vivo Vaccination Studies

Groups of BALB/c mice were injected with unpulsed DCs, soluble β-gal pulsed DCs, PDTP-β-gal DCs or PBS. All of the DCs were exposed to 1 μg/ml CpG 1826 to induce maturation. A minimum of seventeen days after immunization the mice were challenged with a subcutaneous injection of β-gal expressing CT26.CL25 tumor cells or the parental CT26.WT tumor cells. The tumors were measured weekly to determine growth rate and the mice were monitored closely for signs of morbidity. Mice were sacrificed when any dimension of their tumor reached or exceeded 2 cm in size.

Statistical Analysis

Statistical analysis was performed using the Microsoft Excel 97 SR-2 computer program. Error bars in the graphs represent one standard deviation from the mean. P-values were calculated using a two-tailed Student's T test for two samples of equal variance.

Results

Dendritic Cell Biology: Phenotype, Maturation, and Function

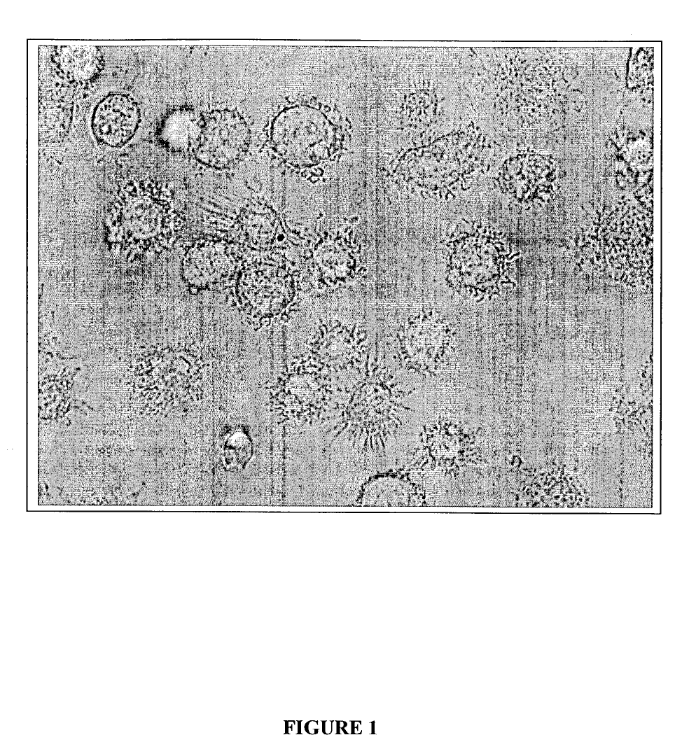

GM-CSF cultured Bone marrow cells have the morphological characteristics of dendritic cells. One of the hallmarks of a dendritic cell is its unique morphology. Studies conducted by Steinman in the early seventies described cells that had an unusual dendritic shape with continually forming and retracting processes (Steinman and Cohn 1973, Steinman and Cohn 1974). When the technology to generate DCs from bone marrow was perfected the cells obtained were described similarly as possessing a distinct dendritic cell shape with sheet like processes or veils (Inaba et al. 1992). To demonstrate DC-like morphology, bone marrow cells were cultured in complete medium containing 200 U/ml rmGM-CSF as described in the methods section. On day 10 of the culture the cells were removed, washed, and placed on a lysine coated glass slide. The cells were then examined microscopically. As shown in FIG. 1, the cells obtained from GM-CSF stimulated bone marrow cell cultures exhibit the “veiled” dendritic cell morphology with the attached cells having the stellate shape that is typical of DCs plated in this manner (Sallusto et al. 1995).

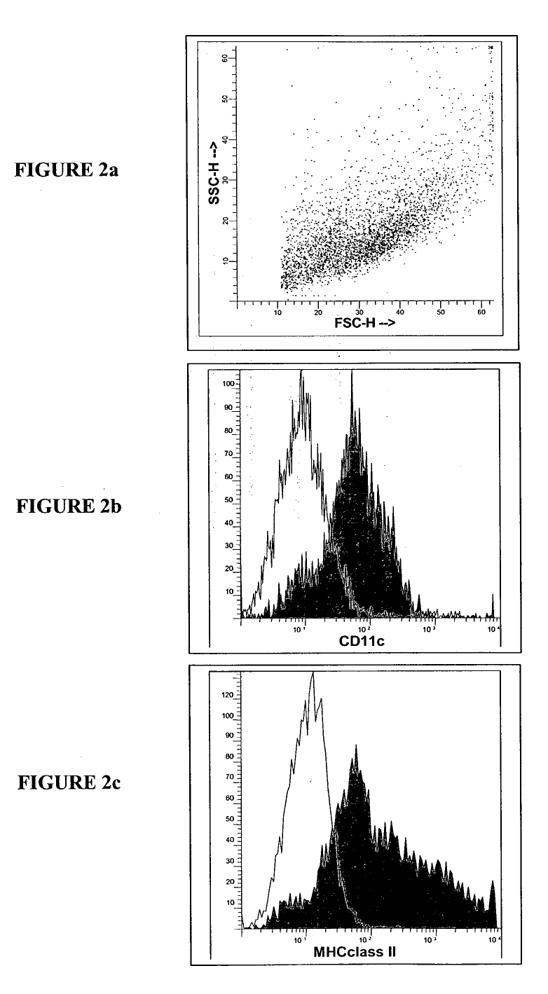

GM-CSF cultured Bone marrow cells have a DC phenotype as determined by FACS analysis. The phenotype of bone marrow derived DCs can be monitored through the use of flow cytometry. Cells recovered from the cultures on day 7 were washed and subjected to analysis by flow cytometry. By side scatter (SSC) and forward scatter (FSC) analysis the non-adherent fraction of the cultured cells displayed the low granularity (SSC) and variable cell size (FSC) typical of bone marrow derived DCs (Lutz et al. 1999) (FIG. 2a). Phenotypic markers characteristic of immature DCs were also examined. The expression of moderate levels of CD11c (FIG. 2b) and MHC class II antigens (FIG. 2c) found by immunophenotyping are indicative of immature DCs (Inaba et al. 1992, Lutz et al. 1999).

Maturation of bone marrow derived DCs. Another defining feature of bone marrow derived DCs is a well-defined cellular response to a maturation signal. Upon maturation, bone marrow derived DCs upregulate surface expression of costimulatory molecules. This upregulation can be quite marked, with the surface expression levels of the costimulatory molecule CD86 increasing up to 100 fold (Mellman and Steinman 2001). Maturation is also accompanied by an increase in the expression level of MHC class II. Flow cytometric analysis of these surface molecules allows for verification of the maturation state of a DC. Two molecules that are known to induce maturation of DCs were used. The bacterial products lipo-polysaccharide (LPS) and a bacterial DNAderived, immunostimulatory oligonucleotide that contains unmethylated CpG repeats are both known to induce DC maturation (Roake et al. 1995, Jakob et al. 1998).

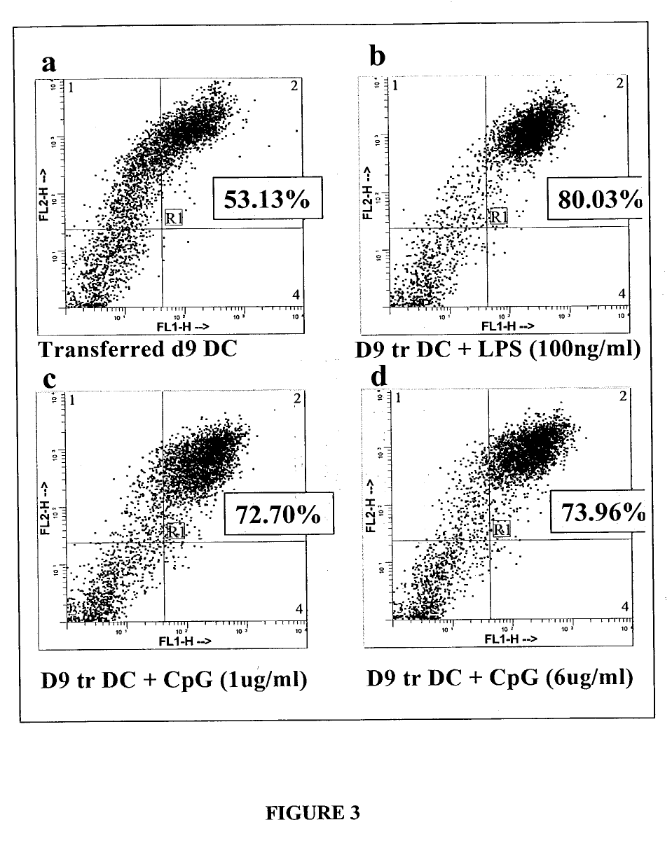

The BM cells were first cultured for 9 days, as outlined above. On the ninth day the cells were collected and replated in a six well culture dish. Cells in separate wells received complete medium containing LPS (100 ng/ml), CpG 1826 (1 μg/ml or 6 μg/ml) or complete medium alone. Sixteen hours later the cells were recovered, washed, labeled and assayed by flow cytometry for expression of MHC class II and CD86. In accordance with earlier findings (Inaba et al. 1992, Lutz et al. 1999) MHC class II and CD86 expression was increased in these cells by addition of LPS or CpG (FIG. 3). Simply moving the cells to a tissue culture treated vessel gave some evidence of maturation, which was lower than that seen with LPS or CpG. This transfer related maturation has been attributed to the production of TNFα by the adherent fraction of cells (Lutz et al. 1999). The increase in the expression level of MHC class II molecules and the appearance of CD86 on the surface of the cultured cells is indicative of not only the identity of these cells as DCs and of their functional ability to respond to two traditionally accepted and evolutionarily important maturation signals.

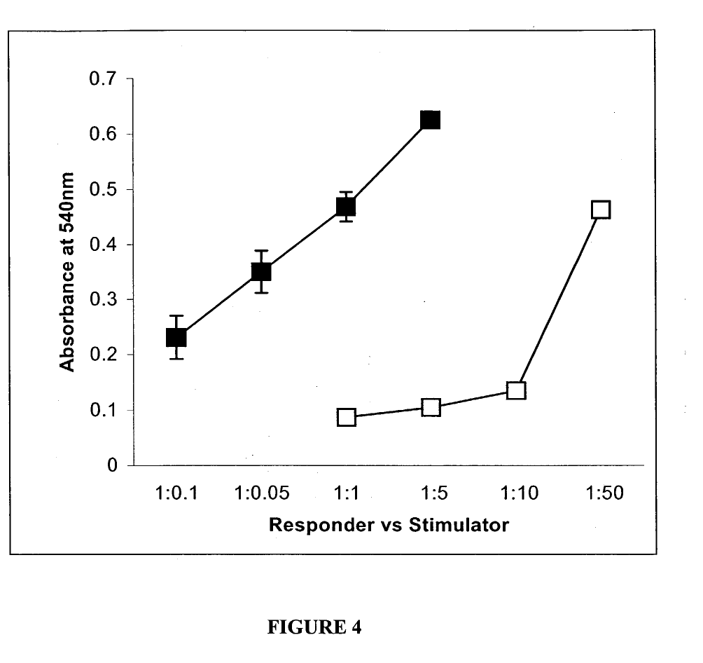

Functional capacity of Bone marrow derived dendritic cells. Upregulation of MHC class II and CD86 in response to bacterial products is one measure of the maturation of DCs. While the response to maturation stimuli is an important step in conferring a functional capacity on DCs, the most common, and it has been argued, the most significant, measure of DC function is the ability to stimulate a one way mixed leukocyte reaction (MLR) (Caux et al. 1999). In an MLR, DCs are at least 100 times more efficient than any other APC in activating T cells (Steinman 1999. This assay has become so important that now high stimulatory activity in the MLR is used to define a DC functionally. BALB/c mouse BM-DCs were generated as described in the methods section. Allogeneic T cells from C57Bl/6 mice were added at 1×10⁵ cells per well and were mixed with varying doses of irradiated treated BM-DCs or splenoctyes. After a three-day incubation period, cell activity (cell number equivalent) was determined by an MTT assay (Mossman 1983, Maghni et al. 1999) as outlined in the methods section. In these experiments the BM-DCs were found to be at least 50 times more efficient than BALB/c splenocytes in stimulating an MLR (FIG. 4). These results were repeated twice more with essentially the same results, showing that the BM-DCs were indeed potent stimulators of an MLR.

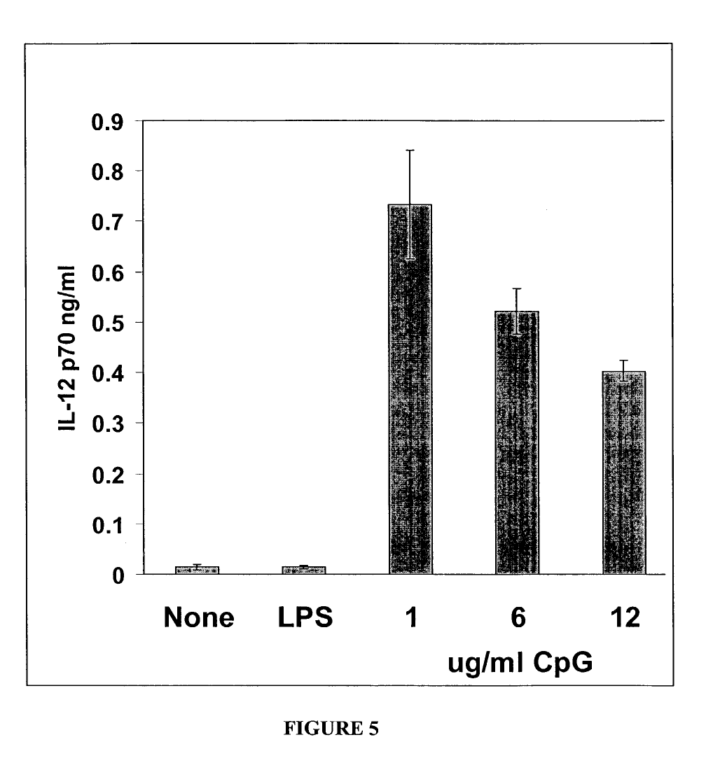

Bone marrow derived DCs produce IL-12 in response to bacterial DNA oligonucleotides that contain unmethylatedCpG motifs. Potent stimulation of T cells in an MLR is considered the most convenient assay for demonstrating the ability of mature DCs (Inaba et al. 1987, Boog et al. 1988) to stimulate T cell proliferation. Yet another, very important aspect of DC activity is the delivery of a so-called third signal (Kalinski et al. 1999). A DC supplies three signals to a naïve T lymphocyte. The first signal gives antigenic information by means of a peptide in the context of an MHC molecule. The second signal provides costimulation that is a gauge of the “danger” concomitant with that antigen. The third signal provides information on the type of antigen present and directs the polarization (T_{H}1 vs. T_{H}2) of the primary T-cell response. The MLR allows for the assessment of the first two signals; allogeneic MHC provides the first signal and the costimulatory molecules present on the DC provide signal 2. The potential for delivering signal three can be measured by monitoring DC production of the T_{H}1 biasing cytokine IL-12. Production of the p70 heterodimer IL-12 by DCs is clearly correlated with sensitization of T_{H}1 lymphocytes in vitro and in vivo (Hilkens et al. 1997, Trinchieri, 1998). Bacterial DNA containing immuno-stimulatory CpG motifs has been shown to bring about the production of IL-12 by DCs (Jakob et al. 1999). An ELISA to detect IL-12 was used to test the ability of the GM-CSF cultured BM cells to respond to CpG oligonucleotides. Day 9 GM-CSF BM culture cells were split into 5 groups and transferred to a tissue culture treated 12 well plate. The cells were transferred in 500 μl of complete medium with 200 U/ml GM-CSF at a concentration of 5×10⁵ cells/well to duplicate wells. The first group received no additional factors. The remaining groups were treated with 500 μl medium containing LPS (200 ng/ml), or varying doses of CpG-1826 (Chu et al. 1997), viz. 2 μg/ml, 12 μg/ml, or 24 μg/ml. The cells were incubated overnight at 37° C. with 5% CO₂, and then the supernatant fluids were assayed for the production of IL-12 by ELISA. Neither transfer of the cells to medium alone nor transfer of the cells to medium containing LPS induced IL-12 production (FIG. 5). Increasing amounts of CpG did not cause the production of greater amounts of IL-12 (p70). Previous reports have shown that the optimal concentration of CpG used to elicit IL-12 production in vaccines and a DC-cell line culture was 6 ug/ml. In my experiments using concentrations greater than 1 μg/ml of CpG actually caused a significant reduction (p≦0.03) in the production of IL-12 p70 (FIG. 5). The observation that increasing the concentration of CpG led to a decrease in IL-12 production necessitated the titration of the amount of CpG that would be used in the maturation of the DCs.

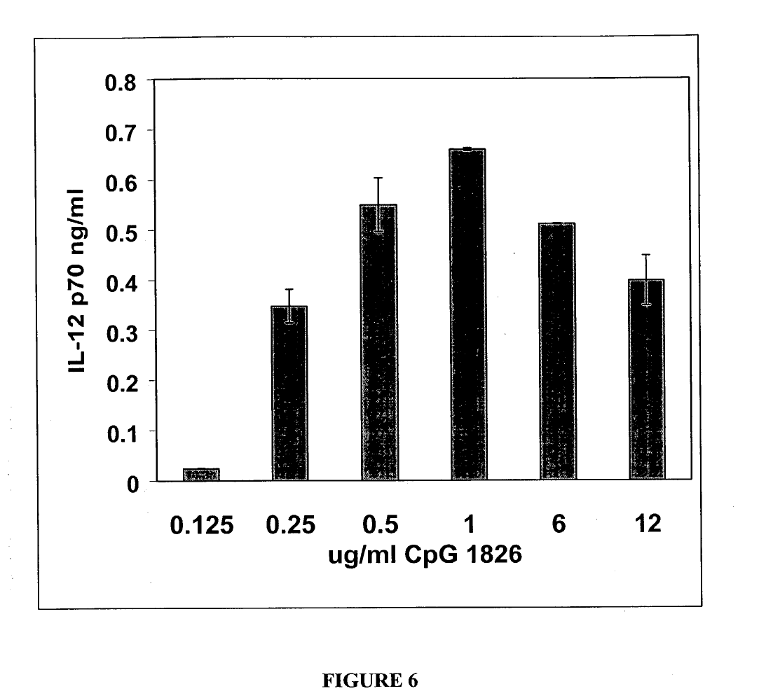

Titration of CpG dose reveals the optimal concentration for the induction of DC derived IL-12. To address the issue of CpG dosage the above experiment was repeated using a wider concentration range of CpG-1826. Groups of DCs were incubated overnight with from 0.125 to 12 μg/ml of CpG-1826. As seen previously doses higher than 1 μg/ml led to decreased IL-12 production (FIG. 6). Reducing the concentration of CpG-1826 in the overnight cultures revealed that the optimal dose for the production of IL-12 (p70) was 1 μg/ml. Using the dose of 1 μg/ml CpG 1826 will not only allow for maximal IL-12 release by these cells but will also induce their maturation as seen by the upregulation of MHC II and CD86 displayed in FIG. 3. The experiments using the CpG-1826 establish that the cells derived from the 9 day in vitro culture in GM-CSF are able to respond to bacterial DNA in a manner that is consistent with their characterization as dendritic cells and consistent with their ability to provide a T_{H}1 biased third signal.

The cells described here are morphologically, phenotypically, and functionally dendritic cells. The cells possess the morphological characteristics and the CD11c and MHC class II expression that is typical of DCs. Also consistent with their identity as DCs, the cultured cells upregulate the costimulatory molecule CD86 and increase the levels of MHC class II molecules on their cell surface upon stimulation with maturation factors like LPS and CpG. A high level of T-cell stimulation in the allogeneic MLR confirms that these cells possess the functional, and immunostimulatory capabilities of DCs. Finally, when cultured in the presence of bacterial CpG oligonucleotides, the cultured cells have the ability to produce IL-12, a cytokine that is crucial to the elicitation of a T_{H}1 response to a given antigen. The ability to culture and isolate functional DCs provides an opportunity to test the efficacy of the covalent linkage of Ag to proteins on the surface of DCs as a vaccine for cancer immunotherapy and to compare this novel strategy of DC Ag loading to conventional Ag loading protocols. In the following experiments, β-galactosidase was used as a model tumor specific antigen.

Selection of a tumor antigen: β-galactosidase and its modification by SPDP β-Galactosidase (β-gal) is a bacterial enzyme that is known to induce a T cell mediated immune response when presented by DCs and has been used as a surrogate tumor antigen in investigations into the treatment of cancer in laboratory animals (Wang et al 1995, Irvine et al 1996, Paglia et al. 1996, Specht et al. 1997, and Brunner et al 2000). Vaccinating mice with soluble β-gal pulsed murine BMDCs evokes a protective anti-tumor response in 40% of the vaccinated mice (Paglia et al. 1996,). While the anti-tumor immunity seen in these reports was promising, the protective response to tumor challenge following vaccination was incomplete. This experimental system offered a working model in which to test a novel Ag loading strategy i.e. the covalent antigen conjugation to the surface proteins on dendritic cells. The efficacy of β-gal conjugated DCs as a tumor vaccine could be compared to that of soluble β-gal pulsed DCs.

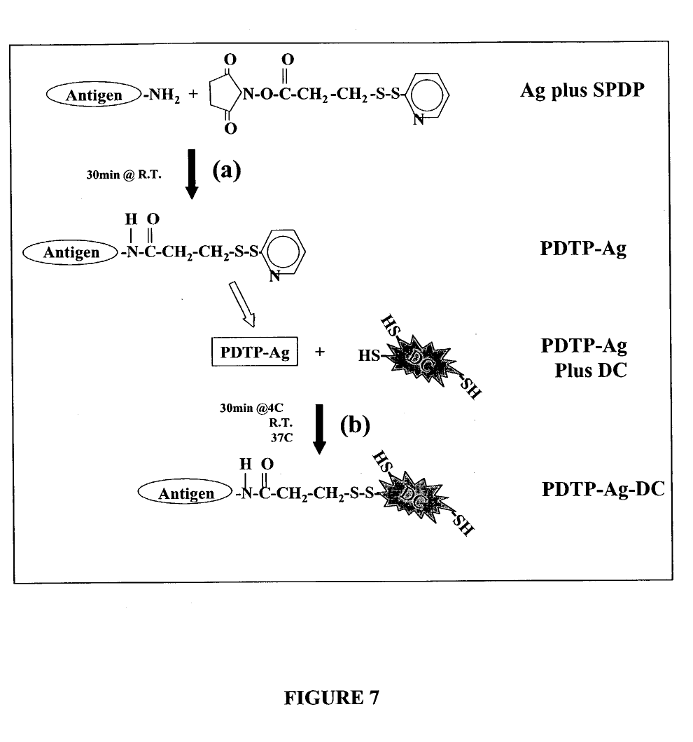

The heterobifunctional reagent, N-succinimidyl 3-(2-pyridyldithio)propionate (SPDP), was selected to link antigens to the plasma membrane proteins on the surface of DCs through the creation of a disulfide bond. Heterobifunctional reagents such as SPDP, link to the protein in the first reaction and then link to the surface membrane protein in the second reaction. These reactions are performed in separate sequential steps, in a process that allows the two reactive groups of SPDP to react with two different targeted functional groups and thereby avoids cross-linking of antigen molecules (FIG. 7).

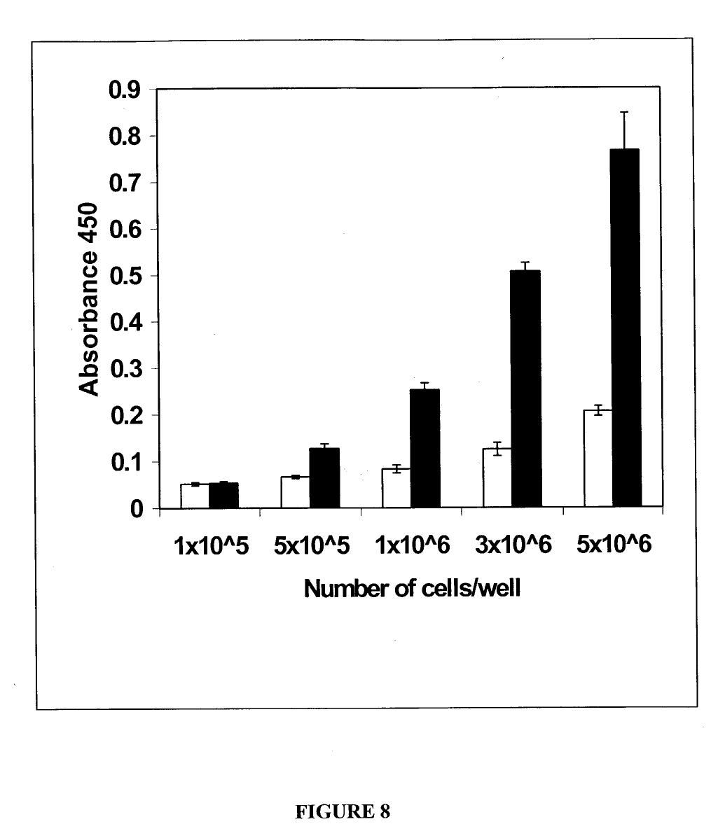

Binding of SPDP modified β-Gal to cell surface-increased plasma membrane associated enzyme activity. To determine if the SPDP modified β-gal linked to cell surface proteins and to reveal the amount of Ag present on the surface of the DC, the enzyme activity of β-gal on the surface of the DCs incubated with the modified Ag was determined. DCs were incubated for 1 hour at 4° C. with either soluble β-gal or PDTP modified β-gal and the cells were washed and then added to wells containing a substrate solution. FIG. 8 shows that DCs incubated with PDTP-β-gal exhibited enzyme activity and that the level of β-gal was significantly greater than that which was observed on DCs pulsed with soluble β-gal. Some residual soluble β-gal binding on the cell surface of the DCs was observed. This was most likely due to multilectin receptors on the DCs that bind the carbohydrate moieties of the glycosylated β-gal. The possibility that the binding of PDTP-β-gal may be due to an increase in non-covalent association is unlikely because extensive washing with PBS and BSA should remove the majority of a non-specifically bound protein.

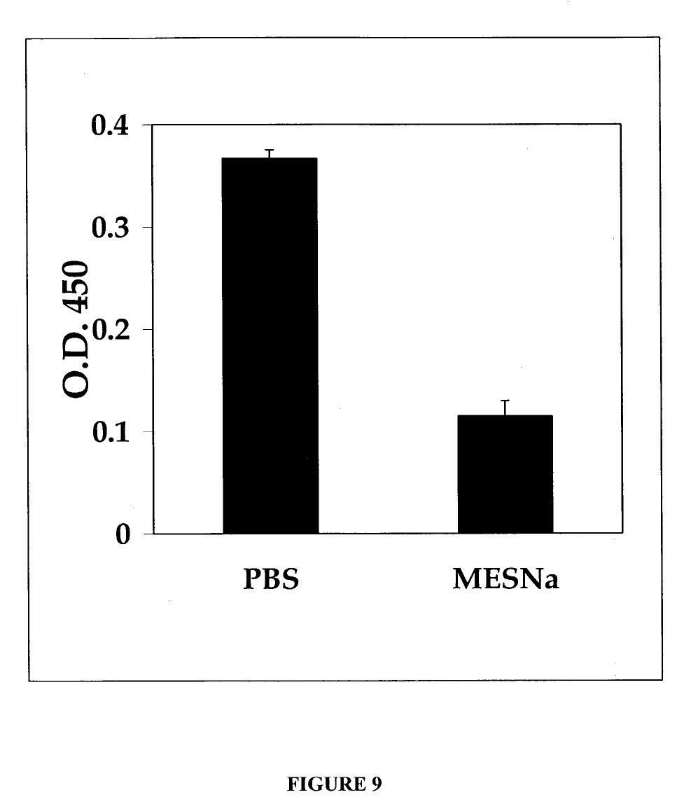

Evidence that PDTP-modified β-Gal binds covalently to the cell surface proteins on DCs. PDTP modified proteins should bind to the surface proteins of a cell through the formation of a covalent disulfide bond. If this occurs then cleavage of the newly formed disulfide bond would liberate β-gal from the cell surface and lead to a reduction in the β-gal activity observed on the surface of the DCs. Mercaptoethanesulfonic acid sodium salt (MESNa) was used to selectively cleave disulfide bonds present in proteins on the surface of the DCs. The DCs were incubated for 1 hour at 4° C. with PDTP modified β-gal and the cells were washed, with one group of cells being exposed to three rounds of MESNa treatment as outlined in the methods section. Both groups of cells were then washed extensively before being added to wells containing a substrate solution. FIG. 9 demonstrates that the increased binding seen with the PDTP-β-gal pulse could be reversed after treating the cells with MESNa. Cleavage of the disulfide bonds created by the interaction of PDTP-β-gal with free thiol groups on the surface of the DCs resulted in a 68.7% reduction of β-gal enzymatic activity i.e. to levels equal to those seen with DCs pulsed with a soluble β-gal. This finding supports the assumption that PDTP-modified β-gal is covalently linked to the surface of the DCs. As expected, treatment of soluble β-gal pulsed DCs with MESNa did not significantly alter the β-gal surface activity on these cells (data not shown). Removal of covalently linked β-gal by MESNa also led us to design a protocol that made it possible to determine whether the Ag that was covalently linked to surface proteins could be internalized by the DCs.

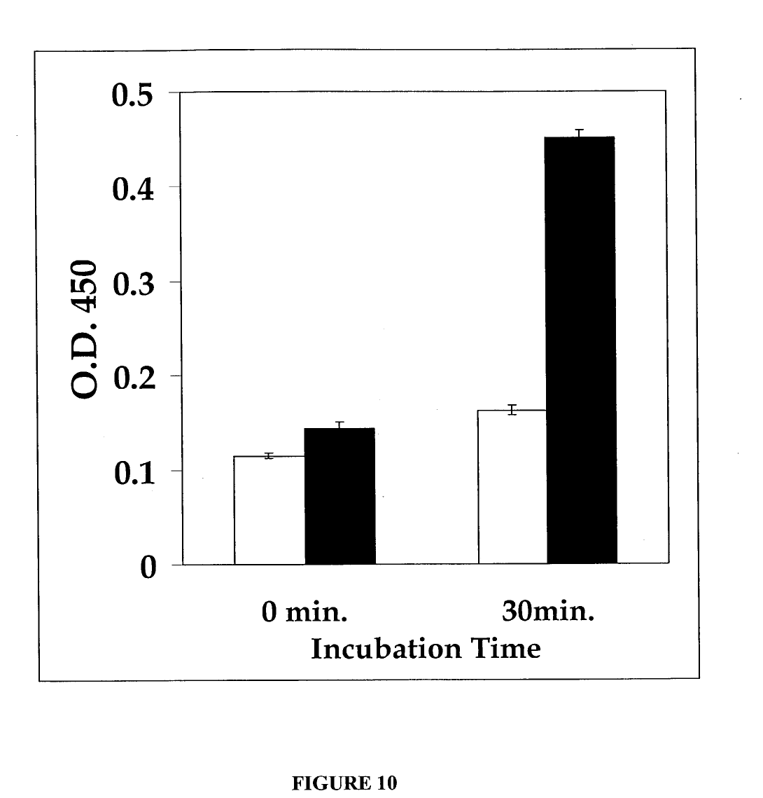

β-Gal covalently conjugated to DCs is internalized. To determine if the β-gal that was linked to the cell surface proteins of DCs, was internalized, PDTP-β-gal loaded DCs were incubated at 37° C. for 0 and 30 minutes and the surface β-gal cleaved with MESNa. The DCs were split into two different groups at each time point. The cells were then washed thoroughly. Half of the cells from each of the 0 and 30 minute incubation time points were subjected to freeze thaw lysis and half were left as intact cells. The cells or an equal amount of cell equivalents in lysis supernatant was then assayed for β-gal activity. FIG. 10 shows that a significant amount of β-gal was protected from cleavage with MESNa as it was found in the cell lysate after a 30 minute incubation at 37° C. This is consistent with the theory that a significant portion of the enzyme linked to the surface was internalized constitutively just 30 minutes after the initiation of a 37° C. incubation period. No evidence of internalization was observed during a 30-minute incubation period at 4° C. (data not shown). It can be seen that antigen can be covalently linked to the surface proteins of DCs and those cells can internalize that antigen. The β-gal that is loaded on DCs through covalent linkage is believed to be degraded after internalization. Proteins degraded by APCs such as DCs are presented as peptides associated with surface MHC molecules.

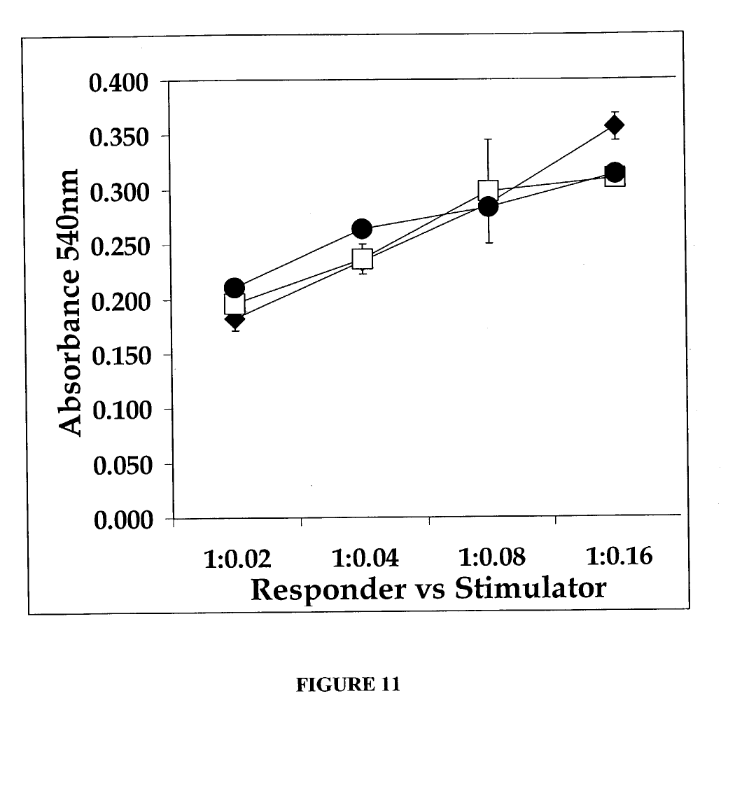

Covalent coupling of β-Gal to DCs does not alter the cell's functional ability to stimulate naïve T cells in an MLR. It was important to determine whether the covalent linkage of β-gal to the surface of the DC would change the cell's stimulatory capacity in a one-way MLR. To this end DCs were cultured overnight in the absence of Ag, with soluble β-gal or with PDTP β-gal. After washing away unbound Ag, increasing numbers of irradiated DCs were mixed with C57Bl/6 mouse lymphocytes. There was no measurable difference in the ability of any of the preparations (soluble β-gal pulsed DCs, PDTP-β-gal pulsed DCs or unpulsed DCs) to stimulate an allogeneic MLR (FIG. 11). The MLR data established that the covalent coupling of an Ag to the surface of DCs does not adversely affect the cell's capacity to stimulate an immune response.

Covalent coupling of β-Gal to DCs does not alter the cells' ability to secrete cytokines in vitro. In addition to testing the functional capacity of the antigen-conjugated cells in the MLR, the effect of covalent linkage of Ag to DCs on the cells' capacity to secrete cytokines in response to a maturation signal were examined. DCs were first pulsed overnight with soluble β-gal or PDTP-β-gal or left unpulsed. Medium that was added the next day was standard medium that contained CpG 1826 (1 μg/ml). A flow cytometric bead ELISA was used to determine the amounts of IL-1β, IL-6, and TNFα released from unpulsed DCs, soluble β-gal pulsed DCs and β-gal-conjugated DCs. Table 1 shows the results of the cytometric bead ELISA.

TABLE 1
Flow Cytometric Bead ELISA Analysis of DC Cytokine Secretion
Values are the mean of two samples in pg/ml +/− one standard deviation

| | IL1-β | IL-6 | TNFα |
|---|---|---|---|
| Unpulsed DC | 503 +/− 42 | 15184 +/− 1277 | 16050 +/− 501 |
| Soluble β-gal | 1594 +/− 172 | 23972 +/− 3116 | 20944 +/− 3251 |
| Pulsed DC | | | |
| PDTP β-gal | 1460 +/− 289 | 25983 +/− 1648 | 21691 +/− 3666 |
| Pulsed DC | | | |

There were some significant differences in the amount of cytokines released by unpulsed DC and the two β-gal pulsed DC groups (IL-β p≦0.043 and IL-6 p≦0.02) between unpulsed. However, there were no significant differences in the cytokines produced by soluble β-gal+CpG pulsed DCs or PDTP-β-gal+CpG pulsed DCs. There was also no impairment of the ability of the cells in the PDTP-β-gal pulsed cell culture to produce IL-1β, IL-6 or TNFα.

Having established that the covalent coupling of Ag to DCs had no demonstrable effects upon cell viability and function, it was determined if the covalent antigen loading of DCs would convey upon these cells an enhanced capacity to induce an anti-tumor immune response. To test and compare the immunogenic capacity of the different DC groups a tumor model with a well-defined tumor specific Ag (β-gal) was selected. The induction of immunity to β-gal was determined by measuring the growth of a β-gal expressing tumor in mice given unpulsed DCs, soluble β-gal pulsed DCs or PDTP-β-gal pulsed DCs.

Comparison of DCs Pulsed with Soluble Tumor Ag to DCs Covalently Linked to Tumor Ag for the Induction of Tumor Immunity in vivo

Colon 26 is a N-nitroso-N-methylurethane induced colon carcinoma established in a BALB/c mouse. This tumor is poorly immunogenic and grows progressively in animals after subcutaneous or intravenous injection (Wang 1995). The tumor has been transfected with the bacterial lac-Z gene, which causes the cells to express β-galactosidase that serves as an experimental tumor antigen. Because there are many assays to detect the presence and activity of β-gal, the ease of detection of this model tumor antigen has made it a popular choice for immunological investigations. Pulsing of DCs with the soluble form of the model tumor Ag β-gal is an established practice in experimental immunotherapy.

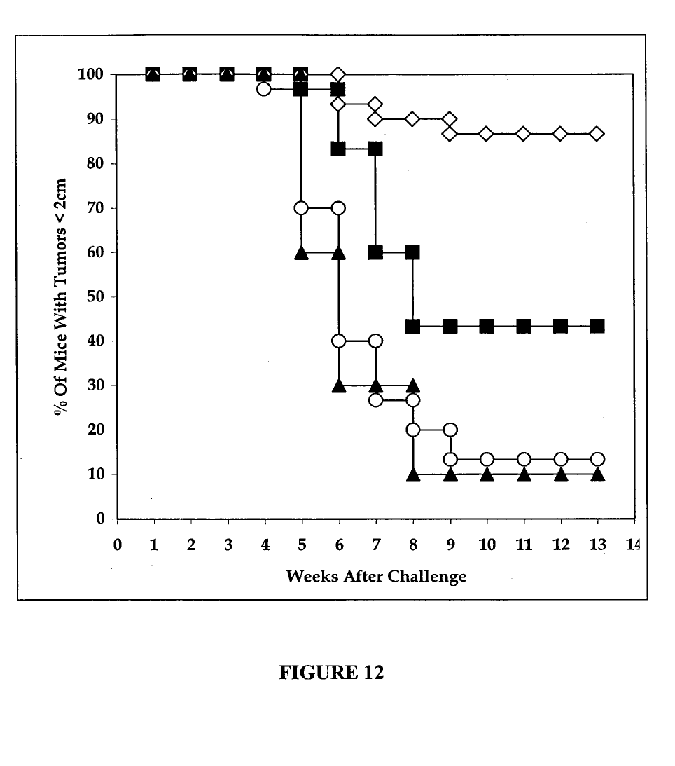

PDTP-β-galactosidase pulsed DC vaccination is superior to soluble β-galactosidase pulsed DC vaccination in protecting mice from challenge with a β-gal expressing tumor. The first step in establishing the efficacy of an anti-tumor vaccine is to establish its ability to induce protective immunity in vivo. Accordingly, groups of BALB/c mice were injected with unpulsed DCs, soluble β-gal pulsed DCs, PDTP-β-gal DCs or PBS. All of the DCs were exposed to 1 μg/ml CpG 1826 to induce maturation. Seventeen days after a single immunization the mice were challenged with a subcutaneous injection of β-gal expressing CT26.CL25 tumor cells. The tumors were measured weekly to determine growth rate and the mice were monitored closely for signs of morbidity. Mice were sacrificed when any dimension of their tumor reached or exceeded 2 cm in size. Six experiments in all were performed using the original vaccination protocol (5×10⁵ DCs injected intraperitoneally). In all six of these experiments mice receiving DCs covalently linked to β-gal had the highest survival rate of the three vaccine preparations. Therefore data from all six experiments were presented together in FIG. 12, which plots the percent survival of 30 mice per treatment group (soluble β-gal and PDTP-β-gal vaccinated and unvaccinated) for the in vivo protection trials. Mice were considered “cured” if the tumor free condition persists for 90 days. Cures were attained in 13.3% of mice vaccinated with unpulsed DCs, 43.3% of soluble β-gal pulsed DC vaccine recipients, and 90.0% of PDTP-β-gal pulsed DC vaccinated animals. The results presented in FIG. 12 reveal a highly significant advantage to vaccination with antigen conjugated DCs when compared to DCs co-incubated with soluble β-gal (p≦0.00011).

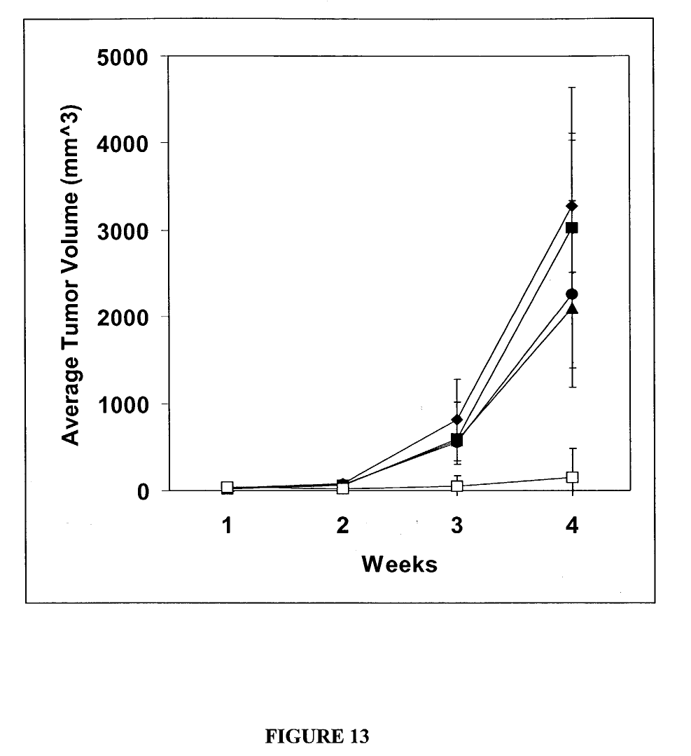

The anti-tumor protection provided by DC vaccination is Ag specific. Vaccinating mice with DCs, and then challenging them with β-gal negative CT26.WT cells addressed the issue of specificity. The vaccinations were carried out using the established protocol of 5×10⁵ DCs per mouse. The four vaccination groups consisted of unvaccinated mice or vaccination with unpulsed DCs, soluble β-gal pulsed DCs, or PDTP-β-gal pulsed DCs. The growth of non β-gal expressing CT26.WT tumors was progressive in all four groups challenged with that parental cell line (FIG. 13). One mouse that was vaccinated with soluble β-gal pulsed DCs did not demonstrate any tumor growth. The DCs prepared for this experiment were capable of protecting mice from challenge with the β-gal expressing CT26.CL25 tumor cells. As shown in FIG. 13, PDTP-β-gal pulsed DCs provided protection from the β-gal expressing CT26.CL25 tumor challenge. These results establish that the protection afforded by the PDTP-β-gal pulsed DC vaccination does not extend to the CT26.WT cell line, suggesting that vaccination of mice with Ag pulsed DCs did not elicit an innate immune response that is strong enough to affect the growth of the non-β-gal transfected parental tumor CT26.WT. The immune response to soluble β-gal pulsed and β-gal-conjugated DCs is specific, as vaccination with these preparations only protect against challenge of a tumor that expresses β-gal. The specificity exhibited here is one of the hallmarks of an adaptive immune response. Another measure of the adaptive immune response is memory.

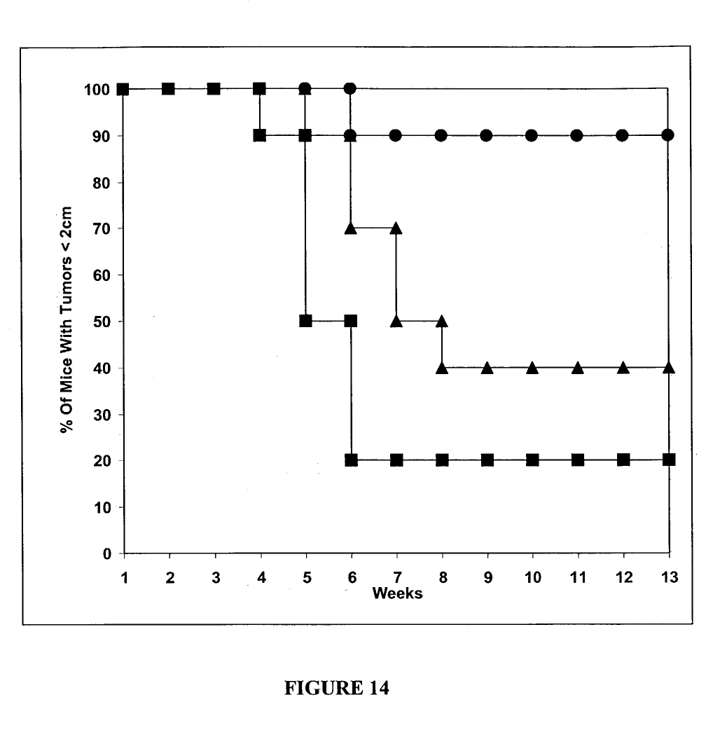

The protection provided by DC vaccination is long lived. To evaluate the induction of memory the tumor challenge was delayed after vaccination. Animals vaccinated up to 7 weeks prior to challenge still maintained the ability to protect 4 of 10 mice vaccinated with soluble β-gal and 8 of 10 mice vaccinated with β-gal-conjugated DCs (FIG. 14). That such a vaccination approach elicits immunological memory further suggests that antigen covalently linked to the DCs induced a greater degree of protection from challenge than that which was observed with DCs pulsed with soluble Ag (p≦0.03). A more clinically relevant issue however, is whether either vaccination protocol has an effect upon established tumors.

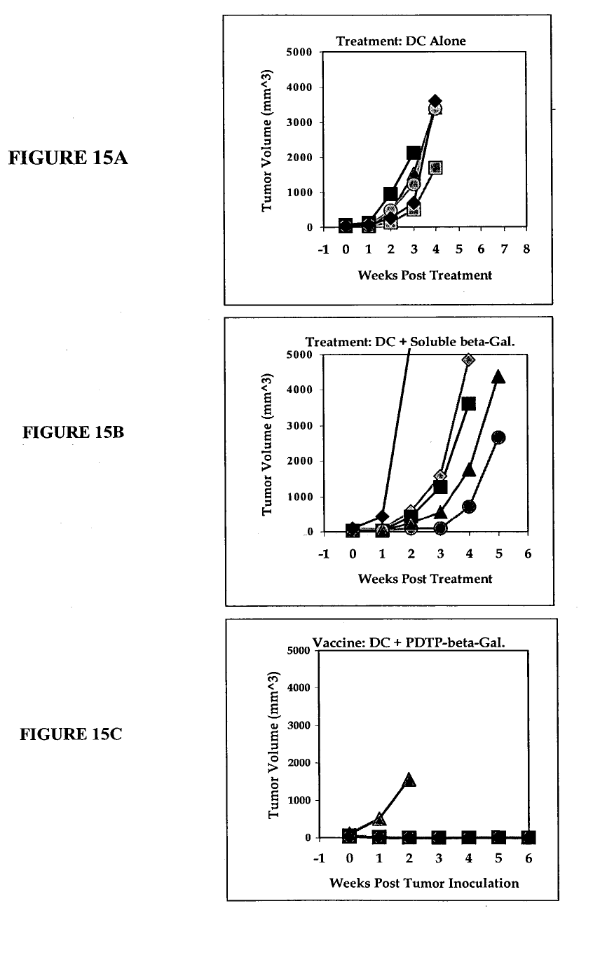

PDTP-β-galactosidase pulsed DCs but not DCs pulsed with soluble β-gal suppress or eliminate established β-gal positive tumors. A therapeutic model was established to test the efficacy of DC vaccine in a more clinically relevant setting. Mice were inoculated with 5×10⁵ CT26.CL25 cells subcutaneously on their left flank. The tumors were allowed to establish and grow for 10 days at which point the mice were redistributed into the three groups that would receive one of three different DC therapies. The tumor sizes were measured and documented on day 10. In the DC only vaccine group the average size of the tumors was 40.29 mm³ (the range being 19 to 50 mm³), mice receiving soluble β-gal pulsed DCs had an average tumor size of 52.30 mm³ (21 to 90 mm³ range), and the third group of animals with an average tumor size of 52.82 mm³ (26 to 104 mm³ range) received DCs with covalently coupled β-gal. The DCs were prepared as in previous experiments, including the addition of 1 μg/ml CpG in the final 6 hours of the in vitro culture. The DCs were washed thoroughly and injected s.c. on the flank opposite the established tumor, i.e. on the right side. The mice were monitored closely and tumor measurements were taken weekly. Mice receiving unpulsed DCs and soluble β-gal pulsed DCs did not respond to the treatment, as evidenced by the continued growth of the established CT26.CL25 (β-gal expressing) tumors (FIG. 15). In contrast, in four of five mice treated with β-gal conjugated DCs, evidence of tumor suppression or complete eradication was observed. In mice treated with PDTP-β-gal loaded DCs rapid involution of the s.c. tumors was observed. Such a rapid anti-tumor effect is suggestive of an innate immune response. In this setting, a clear advantage can be seen in the covalent coupling of antigen to DCs when compared to a soluble pulse of DCs with that same Ag. The evidence presented here establishes that the DCs covalently linked with Ag but not DCs pulsed with soluble Ag are able to suppress aggressively growing tumors. While the mechanism of action involved in this therapeutic effect is yet to be determined, it is clear that antigen conjugated DCs represent a viable therapeutic strategy. These studies show that this strategy is superior to DCs pulsed with soluble Ag in the induction of both protective and therapeutic anti-cancer immunity. To determine if the vaccination protocols induced the activation of T cells, vaccinated mice were sacrificed and their splenocytes assayed for antigen specific activated T cells using an ELISPOT assay.

Demonstration of Ag Specific, CD8⁺ Cells in Vaccinated Mice

After establishing that β-gal covalently linked to the surface of DCs induced an immune response in vivo, the vaccine's ability to activate T cells in an Ag specific fashion was evaluated. It has been suggested that the cytokine secreting potential of a T cell is more indicative of its anti-tumor reactivity than its ability to lyse tumor targets in vitro (Barth 1991). Here an ELISPOT was used to measure the anti-β-gal response of mice vaccinated with different DC preparations by quantifying IFNγ producing T cells in the spleens of vaccinated mice. The DCs were prepared and injected as outlined in the methods section.

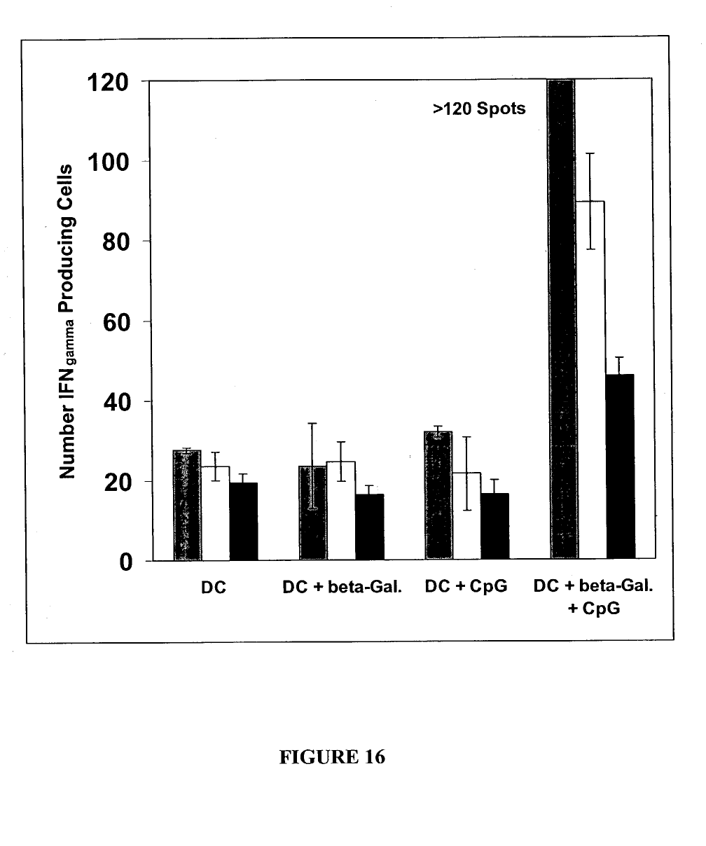

DCs require a maturation stimulus to evoke a response that can be detected in an ELISPOT assay. To examine the effects that maturation has on the Ag pulsed DCs used in these vaccinations, the DCs used in the vaccinations were exposed to CpG 1826 prior to their injection into mice. Four groups of DCs were prepared, two plates each of unpulsed DCs and soluble β-gal pulsed DCs. After an overnight DC incubation period with or without β-gal, one plate of unpulsed DCs and one plate of β-gal pulsed DCs were treated with 1 μg/ml of CpG for 6 hours as a maturation stimulus. The two remaining plates, one plate of unpulsed DCs and one plate of β-gal pulsed DCs received an equal amount of PBS. Following the 6-hour incubation, 5×10⁵ DCs were injected into BALB/c mice. The first group of mice received unpulsed DCs that had been incubated with CpG and the second group received unpulsed DCs that had not been incubated with CpG. In addition, the third group received β-gal pulsed DCs that had been incubated with CpG and one group received β-gal pulsed DCs that had not been exposed to CpG. Twelve days after the vaccination the mice were sacrificed and their splenocytes were restimulated in vitro for 6 days by co-culturing them with irradiated β-gal expressing P13.4 tumor cells at a ratio of 100 splenocytes to 1 tumor cell. The P13.4 cell line is a subclone of the DBA/2 (H-2d) mastocytoma P815 that expresses β-galactosidase (Carbone and Bevan 1990). The cells from the four groups were collected and distributed in two fold dilutions to an ELISPOT plate that had been coated with an anti-IFNγ antibody. The cells were then cultured overnight with the addition of the β-gal expressing P13.4 cell line. The cells were then incubated overnight in the ELISPOT plates after the 20-hour incubation period the ELISPOT assay was performed. The only vaccination strategy to result in the induction of IFNγ producing cells was that which employed the in vitro pulse of DCs with soluble β-gal with addition of CpG (FIG. 16). The vaccination of animals with unpulsed DCs plus CpG did not elicit a response above background showing that the response seen was dependent on Ag and not the result of an nonspecific response to any factor produced by CpG treated DCs. Thus CpG induced maturation of the DCs used in vaccinating mice against β-gal proved to be pivotal in determining the ability to detect the production of IFNγ. With the completion of a successful ELISPOT experiment came the ability to test the relative efficacy of the soluble β-gal and PDTP β-gal loaded DC vaccine preparations.

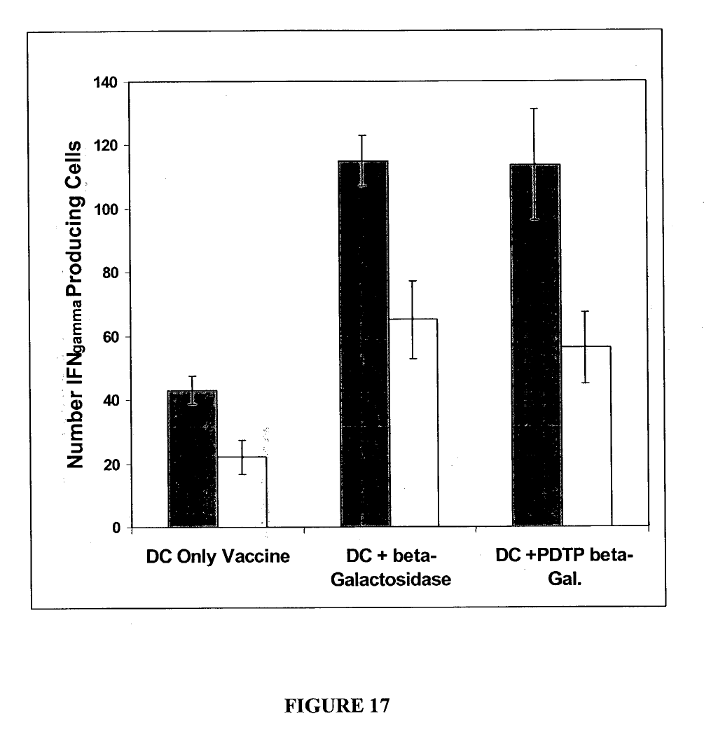

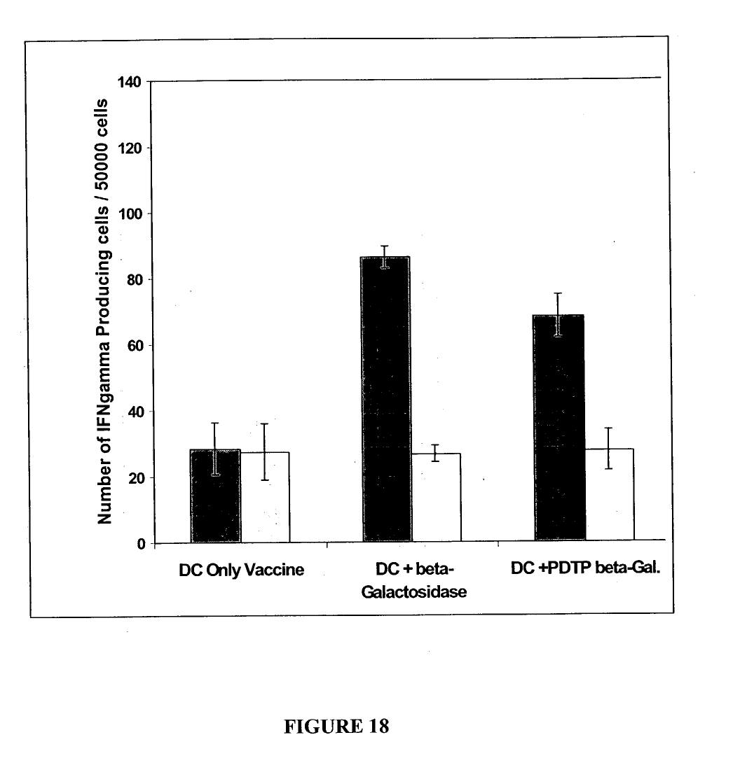

PDTP-β-gal pulsed DCs elicit a β-gal specific response that is equal to the response seen with soluble pulsed β-gal DCs. An in vitro analysis of a the immune response generated by β-gal conjugated DC vaccination and soluble β-gal pulsed DC vaccination was undertaken to compare the ability of the two protocols to elicit IFNγ production in T cells. Mice were vaccinated intraperitoneally with 5×10⁵ unpulsed DCs, soluble β-gal or PDTP-β-gal pulsed DCs (all were matured using CpG 1826). The splenocyte collection and 6 day in vitro restimulation (IVS) was carried out as previously stated with the stimulator cells being irradiated P13.4 cells. After collection, representative cells were then cultured in ELISPOT plates for 20 hours with irradiated, β-gal expressing, P13.4 tumor cells. FIG. 17 demonstrates that mice receiving either of the antigen pulsed DC vaccinations had significantly higher numbers of IFNγ producing cells than did mice receiving unpulsed DCS (p>0.001). However, the number of IFNγ producing cells obtained from mice vaccinated with PDTP-β-gal DCS did not differ significantly from the number obtained from mice vaccinated with soluble β-gal (p=0.9010). No response was observed when cells were incubated without restimulation with β-gal transfected tumor. The results of these experiments establish that the covalent coupling of Ag to the surface proteins of DCs does elicit an Ag specific response but does not result in an increase in the number of β-gal specific T cells as determined by the ELIPSOT assay. These results were repeated twice more with essentially the same results. CD8⁺ cells are responsible for the IFNγproduction in the ELISPOT assay. While the ELISPOT assay shows that the T cell response to the two vaccinations is equivalent, the T cell subset responding to vaccination, as measured by the ELISPOT, is not known. To address this issue CD8⁺ cells were separated after the completion of the 6 day in vitro culture. The CD8⁺ and CD8⁻ cell populations were restimulated overnight in ELISPOT plates with irradiated P13.4 cells. The CD8⁻ fraction of cells displayed background levels of IFNγ producing cells (FIG. 18). The IFNγ response to vaccination was found to be entirely due to the cytokine production by CD8⁺ cells (FIG. 18). The IFNγ producing cell numbers from the soluble and PDTP-β-gal groups were not significantly different (P≦0.0617). These results suggest vaccination with both soluble β-gal pulsed and PDTPβ-gal pulsed DCs elicit an equivalent CD8⁺ T cell response as assessed by IFNγ release.

β-Gal is Required for IFNγProduction in the ELISPOT Assay.

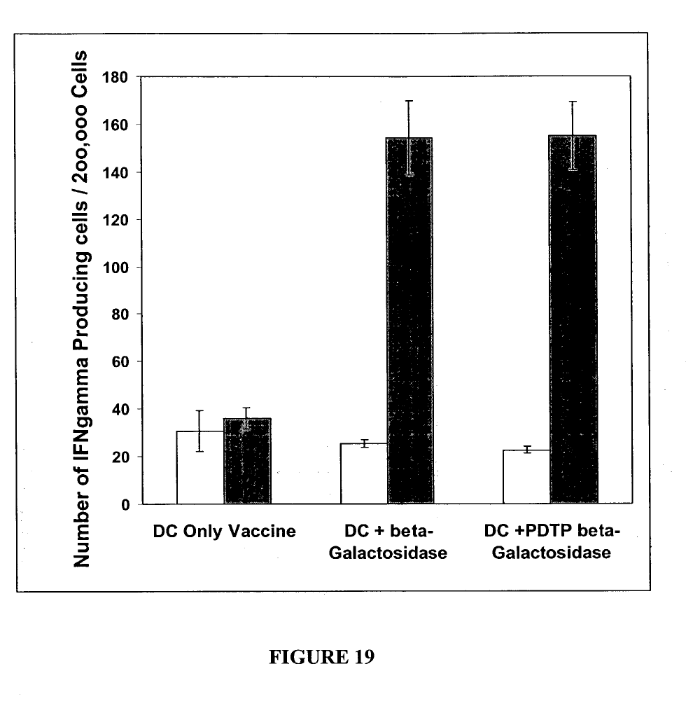

To determine if the response seen in the ELISPOT results is due to a restimulation of immune cells that are specific for β-gal, mice were vaccinated with unpulsed DCs, DCs pulsed with soluble β-gal or DCs pulsed with PDTP-β-gal. The splenocytes from each vaccinated group were split into two groups for restimulation upon recovery. The first restimulation was with co-cultured irradiated, β-gal negative CT26.WT tumor cells and the second restimulation group was co-cultured with β-gal expressing CT26.CL25 cells. Cells that were co-cultured with CT26 produced background levels of IFNγ secreting cells equal to that seen in splenocytes obtained from unpulsed DCs (FIG. 19). Splenocytes recovered from animals vaccinated with β-gal pulsed DCs showed evidence of a response only when restimulated with the β-gal expressing tumor CT26.CL25. The ELISPOT results show that the production of IFNγ is dependent on the presence of β-gal in the 6-day IVS.

The data obtained from all of the in vitro analysis (using the ELISPOT) previously described suggest that DCs covalently linked to Ag are able to induce a T cell response of similar magnitude to that of DCs pulsed with soluble β-gal. In contrast, in the in vivo models described previously, DCs covalently coupled to Ag elicit a more potent anti-tumor immune response than did DCs pulsed with soluble antigen.

Others have also suggested that in vitro assays, such as the ELISPOT and CTL assays do not correlate with the outcome of the overall immune response in vivo (Dallal and Lotze 2000). The production of IFNγ as measured by the ELISPOT assay is only one parameter that can be used as an in vitro measure of a vaccine's efficacy. Therefore a more telling measure of vaccine efficacy can be addressed in murine models by measuring immune activity in vivo, using protection from or treatment of a tumor challenge. In conclusion, the measurement of IFNγ production by restimulated splenocytes may be too narrow a parameter to measure the efficacy of a protective or therapeutic anti-cancer vaccine. In measuring tumor progression in vivo, Ag covalently linked to the surface of DCs has been found to be superior to that of DCs pulsed with soluble Ag in the induction of protective and therapeutic anti-tumor immunity.

### REFFERENCES

1. Albert et al., Journal of Experimental Medicine. 188: 1359-1368, 1998a

2. Albert et al., Nature 392:86-89, 1998b

3. Arnold-Schild et al., Journal of Immunology. 162:3757-3760, 1999

5. Banchereau et al., Annual Review of Immunology. 18:767-811, 2000

6. Barth et al., Journal of Experimental Medicine. 173: 647X-658X, 1991

7. Boog et al., European Journal of Immunology. 18(2):219-23, 1988.

8. Brunner et al., Journal of Immunology. 165(11):6278-86, 2000

9. Carbone et al., Journal of Experimental Medicine. 171(2):377-87, 1990

10. Caux et al., in Dendritic Cells Biology and Clinical Applications, edited by Lotze M. and Thompson A. Academic Press Developmental pathways of human myeloid dendritic cells, 63-92, 1999

11. Celluzzi et al., Journal of Immunology. 160(7):3081-5, 1998

12. Chu et al., Journal of Experimental Medicine. 186(10):1623-31, 1997

13. Dallal et al., Current Opinion in Immunology. 12(5):583-8, 2000

14. Fanger et al., Journal of Immunology. 157:541-548, 1996

15. Fong et al., Annual Review of Immunology. 18:245-73, 2000

16. Gong et al., Journal of Immunology. 165(3):1705-11, 2000

17. Hilkens et al., Blood. 90(5):1920-6, 1997

18. Ignatius et al., Blood. 96(10):3505-3513, 2000

19. Inaba et al., Journal of Experimental Medicine. 166(1):182-94, 1987

20. Inaba et al., Journal of Experimental Medicine. 176(6):1693-702, 1992

21. Irvine et al., Journal of Immunology. 156(1):238-45, 1996

22. Jakob et al., Journal of Immunology. 161(6):3042-9, 1998

23. Jakob et al., International Archives of Allergy & Immunology. 118(2-4):457-61, 1999

24. Kalinski et al., Immunology Today. 20(12):561-7, 1999

25. Kaplan et al., Journal of Immunology. 163(2):699-707, 1999

26. Krieg et al., Current Topics in Microbiology & Immunology. 247:1-21, 2000

27. Labeur et al., Journal of Immunology. 162(1):168-75, 1999

28. Laus et al., Nature Biotechnology. 18(12): 1269-1272, 2000

29. Lutz et al., Journal of Immunological Methods. 223(1): 77-92, 1999

30. Maghni et al., Journal of Immunological Methods. 223: 185-194, 1999

31. Manetti et al., Journal of Experimental Medicine. 177(4):1199-204, 1993

32. Mayordomo et al., Journal of Experimental Medicine. 183(4):1357-65, 1996

33. Moser et al., Nature Immunology. 1(3):199-205, 2000

34. Mossman Journal of Immunological Methods. 65: 55-63, 1983.

35. Mosmann et al., Journal of Immunology. 136(7):2348-57, 1986

36. Mosmann et al., Immunology Today. 17(3):138-46, 1996

37. Paglia et al., Journal of Experimental Medicine. 183(1):317-22, 1996

38. Roake et al., Journal of Experimental Medicine. 181(6):2237-47, 1995

39. Rubartelli et al., European Journal of Immunology. 27:1893-1900, 1997

40. Sallusto et al., Journal of Experimental Medicine. 182(2):389-400, 1995

41. Sher et al., Annual Review of Immunology. 10:385-409, 1992

42. Steinman et al., Proceedings of the National Academy of Sciences of the United States of America. 75(10):5132-6, 1978

43. Steinman, Annual Review of Immunology. 9:271-96, 1991

44. Steinman, Journal of Experimental Medicine. 191(3):411-6, 2000

45. Steinman, in Fundamental Immunology, 4^{th} edition, edited by Paul W. Lippincott-Raven. Dendritic Cells, 547-573, 1999

46. Song et al., Journal of Experimental Medicine. 186(8):1247-56, 1997

47. Sparwasser et al., European Journal of Immunology. 28(6):2045-54, 1998

48. Specht et al., Journal of Experimental Medicine. 186(8):1213-21, 1997 Oct 20.

49. Steinman et al., Journal of Experimental Medicine. 137(5):1142-62, 1973

50. Steinman et al., Journal of Experimental Medicine. 139(2):380-97, 1974

51. Suzue et al., Proceedings of the National Academy of Sciences of the United States of America. 94(24):13146-51, 1997

52. Trinchieri, Blood. 84(12):4008-27, 1994

53. Trinchieri, Advances in Immunology. 70:83-243, 1998.

54. Todryk et al., Journal of Immunology. 163:1398-1408, 1999

55. Vignali, Journal of Immunological Methods. 243(1-2):243-55, 2000

56. Wang et al., Journal of Immunology. 154(9):4685-92, 1995

57. You et al., Cancer Research. 61(1):197-205, 2001

58. Zitvogel et al., Journal of Experimental Medicine. 183(1):87-97, 1996

1. A method for elicting an immune response in an individual to an antigen comprising the steps of covalently coupling the antigen to one or more proteins or glycoproteins on the surface of dendritic cells obtained from the individual and infusing the covalently coupled dendritic cells into the individual.

2. The method of claim 1, wherein the antigen is a protein or peptide from a source selected from the group consisting of tumor, virus and bacteria.

3. The method of claim 2, wherein the antigen is a tumor antigen.

4. The method of claim 1, wherein the covalent coupling is carried out via a heterobifunctional reagent.

5. The method of claim 4, wherein the heterobifunctional regent forms an amide bond at one end and a disulfide bond at the other end.

6. The method of claim 5, wherein the heterobifunctional regent is N-Succinimydyl-3(2-pyridyldithio) propionate.

7. The method of claim 1, further comprising the step allowing the dendritic cells after covalent coupling to the antigen to mature in vitro before reinfusing into the individual.

8. The method of claim 7, wherein the maturation of dendritic cells in vitro is induced by exposure to an agent selected from the group consisting of CpG oligonucleotides, Granulocye, Macrophage Colony Stimulating Factor and Lipopolysaccharide.

9. The method of claim 8, wherein the CpG oligonucleotide has a sequence of SEQ ID NO:1.

10. The method of claim 1, wherein the antigen is purified.

11. The method of claim 1, wherein the antigen is partially purified or unpurified.

12. A composition for eliciting an immune response to an antigen comprising dendritic cells having one or more antigens covalently coupled to one or more proteins on the surface of the dendritic cells.

13. The composition of claim 12, wherein the antigen is a protein or peptide from a source selected from the group consisting of tumor, virus and bacteria.

14. The composition of claim 13, wherein the antigen is a tumor antigen.

15. The composition of claim 12, wherein the antigen is covalently linked to the dendritic cells via a heterobifunctional reagent.

16. The composition of claim 15, wherein the heterobifunctional regent forms an amide bond at one end and a disulfide bond at the other end.

17. The composition of claim 16, wherein the heterobifunctional regent is N-Succinimydyl-3(2-pyridyldithio) propionate.

18. A method for making a composition for eliciting an immune response to an antigen in an individual comprising the steps of:
a) obtaining dendritic cells from the individual,
b) covalently coupling the antigen to one or more proteins or glycoproteins on the surface of the dendritic cells.

19. The method of claim 18, wherein the covalent coupling is carried out using a heterobifunctional reagent.

20. The method of claim 19, wherein the heterobifunctional regent forms an amide bond at one end and a disulfide bond at the other end.

21. The method of claim 20, wherein the heterobifunctional reagent is N-Succinimydyl-3(2-pyridyldithio) propionate.

22. The method of claim 18, further comprising the step of inducing maturation of the dendritic cells in vitro.

23. The method of claim 22, wherein the maturation of dendritic cells in vitro is induced by exposure to an agent selected from the group consisting of CpG oligonucletodies, Granulocye, Macrophage Colony Stimulating Factor and Lipopolysaccharide.

24. The method of claim 23, wherein the CpG oligonucleotide has a sequence of SEQ ID NO:1.

25. The method of claim 18, wherein the antigen is partially purified or unpurified.

26. A method of reducing the growth of a tumor in an individual comprising the steps of:
a) obtaining a tissue sample from the tumor;
b) obtaining an antigen from the tissue sample;
c) obtaining dendritic cells from the individual;
d) covalently linking the antigen to one or more surface proteins or glycoproteins on the dendritic cells; and
e) reinfusing the covalently linked dendritic cells into the individual.

27. The method of claim 26, wherein the covalent coupling is carried out using a heterobifunctional reagent.

28. The method of claim 27, wherein the heterobifunctional reagent forms an amide bond at one end a disulfide bond at the other end.

29. The method of claim 26, further comprising the step of inducing maturation of the dendritic cells in vitro prior to reinfusing the cells in step e).

30. The method of claim 29, wherein the maturation of dendritic cells in vitro is induced by exposure to an agent selected from the group consisting of CpG oligonucleotides, Granulocye, Macrophage Colony Stimulating Factor and Lipopolysaccharide.

31. The method of claim 31, wherein the CpG nucleotide has a sequence of SEQ ID NO:1.

32. The method of claim 26, wherein the antigen is partially purified or unpurified.

33. A method for reducing the recurrence of tumors in an individual in whom the tumor has been surgically removed comprising the steps of:
a) obtaining a tissue sample from the tumor that has been removed;
b) obtaining an antigen from the tissue sample;
c) obtaining dendritic cells from the individual;
d) covalently linking the antigen to one or more surface proteins or glycoproteins on the dendritic cells; and
e) reinfusing the covalently linked dendritic cells into the individual.

34. The method of claim 33, wherein the covalent coupling is carried out using a heterobifunctional reagent.

35. The method of claim 34, wherein the heterobifunctional reagent forms an amide bond at one end and a disulfide bond at the other end.

36. The method of claim 33, further comprising the step of inducing maturation of the dendritic cells in vitro.

37. The method of claim 36, wherein the maturation of dendritic cells in vitro is induced by exposure to an agent selected from the group consisting of CpG oligonucleotides, Granulocye, Macrophage Colony Stimulating Factor and Lipopolysaccharide.

38. The method of claim 37, wherein the CpG nucleotide has a sequence of SEQ ID NO:1.

39. The method of claim 33, wherein the antigen is partially purified or unpurified.

40. A method for preventing the growth of tumors in an individual comprising the steps of:
a) identifying and obtaining an antigen known to be present in the tumors;
c) obtaining dendritic cells from the individual;
d) covalently linking the antigen to one or more surface proteins or glycoproteins on the dendritic cells; and
e) reinfusing the covalently linked dendritic cells into the individual.


SEQUENCE LISTING (1 sequence)

SEQ ID NO: 1 - DNA, 20 bp, Artificial
  synthetic oligonucleotide; CpG 1826
tccatgacgttcctgacgtt